(19) United States
(12) Patent Application Publication
(10) Pub. No.: US 2026/0210965 A1
(43) Pub. Date: Jul. 23, 2026

(54) (POLY)LABEL SIGNAL ENHANCER

(71) Applicant: Roche Diagnostics Operations, Inc., Indianapolis, IN (US)

(72) Inventors: Joerg Freisler, Wielenbach (DE); Dieter Heindl, Muenchen (DE); Lars Hillringhaus, Königsdorf (DE); Samir Karaca, Taufkirchen (DE); Uwe Kobold, Weilheim (DE); Hannes Kuchelmeister, München (DE); Stefan Weiser, Murnau (DE)

(21) Appl. No.: 19/142,066

(22) PCT Filed: Dec. 20, 2023
(86) PCT No.: PCT/EP2023/086953

§ 371 Date: Jun. 20, 2025

(30) Foreign Application Priority Data
Dec. 21, 2022 (EP) 22215674.7

(51) Int. Cl. G01N 33/58; G01N 33/68

(52) U.S. Cl. CPC G01N 33/58; G01N 33/6848

(57) ABSTRACT

A first aspect of the invention is related to the use of a (poly)label for generating a quantifiable signal for an analyte of interest in mass spectrometry, wherein the (poly)label has the structure (I). In a second aspect, the invention is directed to a process for modifying an analyte of interest for obtaining an increased intensity signal in mass spectrometry. A third aspect of the invention is directed to a method for determining an analyte of interest by mass spectrometry. A fourth aspect of the invention relates to a (poly)label having structure of formula (Ia). In a fifth aspect, the invention is related to a reaction product comprising a polypeptide and a (poly)label having the general structure (III).

A first aspect of the invention is related to the use of a (poly)label for generating a quantifiable signal for an analyte of interest in mass spectrometry, wherein the (poly)label has the structure (I). In a second aspect, the invention is directed to a process for modifying an analyte of interest for obtaining an increased intensity signal in mass spectrometry. A third aspect of the invention is directed to a method for determining an analyte of interest by mass spectrometry. A fourth aspect of the invention relates to a (poly)label having structure of formula (I). In a fifth aspect, the invention is related to a reaction product comprising a polypeptide and a (poly)label having the general structure (III).

### STATE OF THE ART

Mass spectrometry (MS) is a widely used technique for the qualitative and quantitative analysis of chemical substances ranging from small molecules to macromolecules. In general, it is a very sensitive and specific method, allowing even for the analysis of complex biological, e.g. environmental or clinical samples. However, for several analytes, especially if analyzed from complex biological matrices, sensitivity of the measurement remains an issue. Often MS is combined with chromatographic techniques, particularly gas chromatography (GC) and liquid chromatography (LC). Here, the molecule of interest is separated chromatographically and is individually subjected to mass spectrometric analysis. There is, however, still a need of increasing the sensitivity of MS analysis methods, particularly for the analysis of analytes that have a low abundance or when only little materials (such as biopsy tissues) are available.

To increase the signal intensity of analytes such as peptides for LC-MS analysis, the following two general strategies have been demonstrated in literature:
(1) Use of LC mobile phase additives such as dimethyl sulfoxide (DMSO) or ethylene glycol (Hahne et al. 2013).
(2) Chemical derivatization to add a permanent positive charge and/or to increase hydrophobicity of analytes to enhance electrospray ionization (Mirzaei et al., 2006).

Regarding the second option, i.e. the signal enhancement by derivatization, for example, for peptide analysis, derivatization reagents such as quaternary ammonium salts, phosphonium salts, or pyridinium salts have been described. However, the prior art has several shortcomings, for example, regarding mobile phase additives, it was reported that, even if addition of DMSO up to 5% into LC solvents enhances electrospray ionization (ESI) of peptides, the degree of ESI enhancement is sample amount dependent, as well as peptide and instrument specific (Hahne et al 2013). Additionally, continuous use of DMSO in LC sol vents requires frequent cleaning of frontend instrument optics, thereby decreasing robustness of the LC-MS instrumentation and increasing downtime.

With view to derivatization to increase ESI of peptides, it has to be noted that this primarily works on peptides, which do not ionize well in native form (Mirzaei et al., 2006). As outlined by Mirzaei et al., peptides bigger than 500 Da and peptides containing cationic amino acids (histidine, lysine, arginine) do not benefit well from derivatization. This makes the actual benefit of derivatization for peptide analysis very limited because basically all peptides used for LC-MS quantification contain cationic amino acids (arginine or lysine due to trypsin digestion) and they are larger than 500 Da; a peptide with a mass of 500 Da roughly corresponds to 4-5 amino acid length, is nonunique and is therefore useless for protein identification and quantification.

Thus, the problem underlying the present invention was the provision of means and methods for increasing analyte signals in mass spectrometry.

### 1^{st} Aspect-Use of a (Poly)Label for Generating a Quantifiable Signal for an Analyte of Interest

A first aspect of the invention is directed to the use of a (poly)label for generating a quantifiable signal for an analyte of interest in mass spectrometry, wherein the (poly)label has the structure (I)

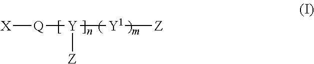

wherein
m is zero or 1;
n is zero or an integer selected from the range of from 1 to 20;
Q is absent or a linker unit;
X is a reactive group or, if Q is absent, a hydrogen atom;
Y is a linker unit based on an amino acid having a side chain suitable for coupling to Z;
Y¹ is a linker unit based on an amino acid having a side chain suitable for coupling to Z, wherein the amino acid has a blocked carboxylic group or a blocked amino acid group;
Z is a moiety selected from the group consisting of
   nucleoside comprising a nucleobase selected from the group consisting of adenine, cytosine, thymine, guanine and uracil;
   tripeptide of structure Z¹-Proline-Z², wherein Z¹ and Z² are independently from each other an amino acid selected from the group consisting of alanine, glycine, valine, leucine, iso-leucine and phenyl alanine; and
   carbamate group —O—C(═O)—NH—(CH₂)_{z}—N⁺(Z³)₃ or —NH—C(═O)—O—(CH₂)_{z}—N⁺(Z³)₃, wherein z is an integer selected from the range of from 1 to 10, and Z³ is a C1 to C5 alkyl group.

In the context of the present invention, a “(poly)label” means a label comprising one or more moieties selected from the above-mentioned group of Z, wherein in case of only one moiety, it is a “label” and in case of ≥2 moieties, it is a “polylabel”.

Contrary to the methods known in the art, which focus on making electrospray ionization of analytes more efficient either by mobile phase additives or by ESI-enhancing chemical derivatization of analytes, the present invention increases the analyte signal by generating multiple copies of the measurand per molecule of analyte. The term “generating a quantifiable signal for an analyte of interest in mass spectrometry” means that a signal is generated in a mass spectrum, which corresponds to the analyte of interest but has a higher intensity compared to the intensity of the molecular ion peak of the analyte of interest and its fragmentation peaks respectively. The quantifiable signal is generated inside of the mass spectrometry, which originates from analyte of interest upon fragmentation. Due to presence of poly-units, the quantifiable signal has either a higher intensity or a higher “fragmentation” efficiency. The “(poly)label” described here comprises one or more quantifier moiety/ies, all being comprised within Z and having the same weight (isobaric), which selectively break(s) apart from the precursor molecule into its single repetitive constituents to generate quantifier ions. Having multiple copies of an isobaric quantifier moiety per analyte increases signal intensity and/or fragmentation efficiency in a very efficient way. The quantifier ions based on the individual (poly)labels of formula (I) are indicated by way of example as follows: For “Z” of formula (I) being, for example, a nucleoside with adenine as nucleobase, the quantifier moiety is adeninine (elemental composition C5H6N5), which generates a (plurality of) quantifier ion(s) having MH⁺¹ of 136 Da. For “Z” of formula (I) being, for example, a carbamate group, the quantifier moiety is a water deprived carbamate group (elemental composition C6H13N20), which generates a (plurality of) quantifier ion(s) having MH⁺¹ of 129 Da. For “Z” of formula (I) being, for example, a tripeptide alanine-proline-glycine (APG), the quantifier moiety is PG (elemental composition C7H12N203), which generates a (plurality of) quantifier ion(s) having MH⁺¹ of 172 Da.

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Percentages, concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “from 4 to 20%” should be interpreted to include not only the explicitly recited values of 4% to 20%, but to also include individual values and sub-ranges within the indicated range. Thus, included in this numerical range are individual values such as 4, 5, 6, 7, 8, 9, 10, . . . 18, 19, 20% and sub-ranges such as from 4-10%, 5-15%, 10-20%, etc. This same principle applies to ranges reciting minimal or maximal values. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “Mass Spectrometry” or “MS” relates to an analytical technology used to identify compounds by their mass. MS is a methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer and an ion detector. In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). The term “ionization” or “ionizing” refers to the process of generating an analyte ion having a net electrical charge equal to one or more electron units. Negative ions are those having a net negative charge of one or more electron units, while positive ions are those having a net positive charge of one or more electron units. The MS method may be performed either in “negative ion mode”, wherein negative ions are generated and detected, or in “positive ion mode” wherein positive ions are generated and detected. “Tandem mass spectrometry” or “MS/MS” involves multiple steps of mass spectrometry selection, wherein fragmentation of the analyte occurs in between the stages. In a tandem mass spectrometer, ions are formed in the ion source and separated by mass-to-charge ratio in the first stage of mass spectrometry (MS 1). Ions of a particular mass-to-charge ratio (precursor ions or parent ion) are selected and fragment ions (or daughter ions) are created by collision-induced dissociation, ionmolecule reaction, or photodissociation. The resulting ions are then separated and detected in a second stage of mass spectrometry (MS2).

While Ionization sources such as Laser desorption ionization (LDI) and atmospheric pressure chemical ionization (APCI) are known, a ionization source preferred in the context of the present invention is electrospray ionization (ESI).

Since a mass spectrometer separates and detects ions of slightly different masses, it easily distinguishes different isotopes of a given element. Mass spectrometry is thus, an important method for the accurate mass determination and characterization of analytes, including but not limited to low-molecular weight analytes, peptides, polypeptides or proteins. Its applications include the identification of proteins and their post-translational modifications, the elucidation of protein complexes, their subunits and functional interactions, as well as the global measurement of proteins in proteomics. De novo sequencing of peptides or proteins by mass spectrometry can typically be performed without prior knowledge of the amino acid sequence.

Mass spectrometric determination may be combined with additional analytical methods including chromatographic methods such as gas chromatography (GC), liquid chromatography (LC), and high-performance liquid chromatography (HPLC), and/or ion mobility-based separation techniques. In the context of the present disclosure, the term “analyte”, “analyte molecule”, or “analyte(s) of interest” are used interchangeably referring the chemical species to be analysed via mass spectrometry. Chemical species suitable to be analysed via mass spectrometry, i.e. analytes, can be any kind of molecule present in a living organism, include but are not limited to nucleic acid (e.g. DNA, mRNA, miRNA, rRNA etc.), amino acids, (poly) peptides, proteins (e.g. cell surface receptor, cytosolic protein etc.), metabolite or hormones (e.g. testosterone, estrogen, estradiol, etc.), fatty acids, lipids, carbohydrates, steroids, ketosteroids, secosteroids (e.g. Vitamin D), molecules characteristic of a certain modification of another molecule (e.g. sugar moieties or phosphoryl residues on proteins, methyl-residues on genomic DNA) or a substance that has been internalized by the organism (e.g. therapeutic drugs, drugs of abuse, toxin, etc.) or a metabolite of such a substance. As outlined also below, preferred analytes of interest are polypeptides and small molecules, more preferably polypeptides. A polypeptide comprises ten or more amino acids, coupled to each other via amide bonds. In case the polypeptide chain contains more than one hundred amino acids and, aside from the primary structure (the polypeptide sequence), also a secondary, tertiary and possibly a quaternary structure are formed, the polypeptide is called a protein. Preferably, a “polypeptide” in the context of the present invention is a peptide wherein in the range of from 2 to 100 amino acids are bound by amide bonds.

Regarding polypeptides as analytes of interest, the (poly)label is, when bound to said polypeptide, bound to the polypeptides C-terminus, N-terminus or to both termini.

In some preferred embodiments of the use of a (poly)label for generating a quantifiable signal for an analyte of interest in mass spectrometry, the (poly)label has a structure of formula (Ia)

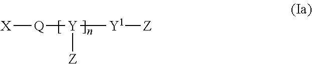

wherein
n is an integer selected from the range of from 1 to 20;
Q is absent or is a linker unit;
X is a reactive group;
Y is a

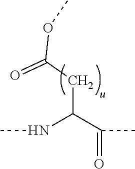

group, or a

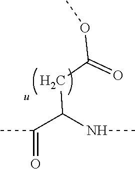

group, wherein the dotted line at the oxygen atom indicates the bond to Z and u is either one or two, or
   a

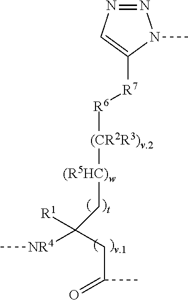

group, or a

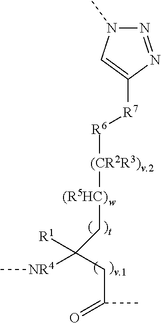

group,
or a

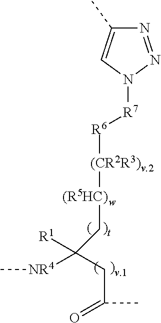

group, or a

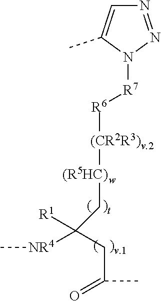

group, wherein the dotted line at the N atom or at the C atom in the triazole ring represent the bond to Z,
t is zero or 1;
v.1 is zero or an integer from the range of 1 to 4;
v.2 is an integer from the range of 1 to 10;
W is zero or 1;
R¹ is a hydrogen atom or a C1-C5 straight or branched alkyl group;
R², R³ are independently from each other and independently for each of the v.2 units selected from hydrogen atom and C1-C5 straight or branched alkyl group;
R⁴ is a hydrogen atom or a C1-C5 straight or branched alkyl group;
R⁵ is a hydrogen atom;
R⁶ is absent or a —C(═O)NH— group or a C(═)O-group or a substituted or unsubstituted C6 to C10 arylene, wherein the one or more substituents are selected from hydrogen atom, halogen atom and functional group;
R⁷ is absent or selected from the group consisting of branched or unbranched C1-C5 alkylene, —O—C1 to C5 alkylene, wherein the C1 to C5 alkylene is branched or unbranched, and —S—C1 to C5 alkylene, wherein the C1 to C5 alkylene is branched or unbranched;
or R⁴ and R⁵ together form a five or six membered heteroalkyl ring, which includes the nitrogen atom of NR⁴ as part of the ring structure;
wherein the dotted line at the NH or NR⁴ for each Y group represents the bond to Q, to the next [Y—Z] unit or to Y¹, the dotted line at the C(═O) for each group represents the bond to the next [Y—Z] unit, to Y¹ or to Q;
Y¹ is a

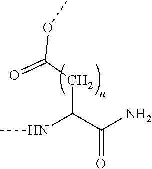

group or a

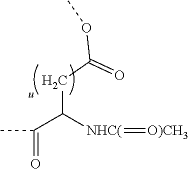

group, wherein the dotted line at the oxygen atom indicates the bond to Z and u is either one or two, or
   a

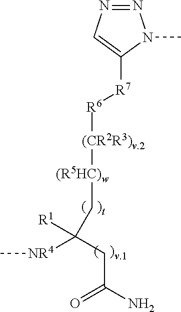

group, or a

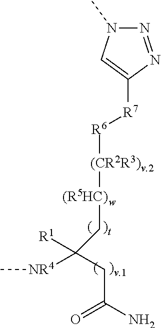

group,
or a

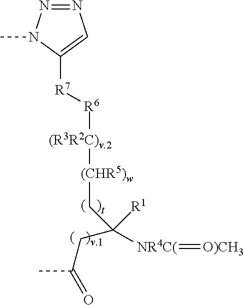

group, or a

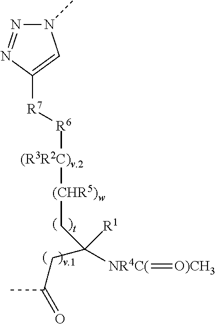

group,
or a

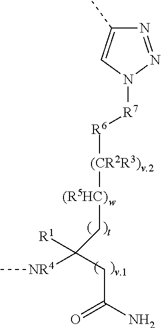

group, or a

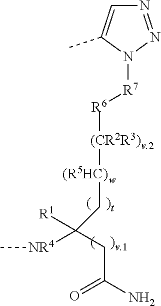

group,
or a

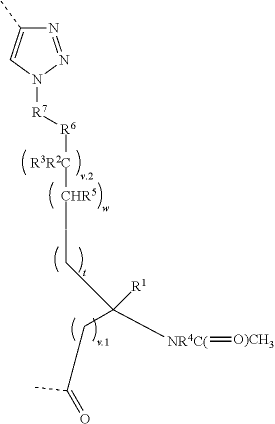

group, or a

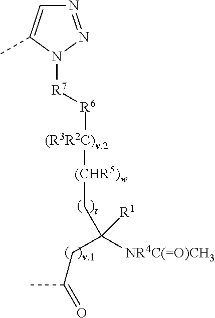

group,
wherein the dotted line at the N atom or at the C atom in the triazole ring represent the bond to Z, and t, v.1, v.2, w and R¹ to R⁵ have the same meaning as above for Y;
wherein the dotted line at the NH or NR⁴ or at the C(═O) for each Y¹ group represents the bond to the next [Y—Z] unit;
Z is a moiety selected from the group consisting of
   nucleoside with a nucleobase selected from the group consisting of adenine, cytosine, thymine, guanine and uracil; and
   carbamate group —O—C(═O)—NH—(CH₂)_{z}—N⁺(Z³)₃, or —NH—C(═O)—O—(CH₂)_{z}—N⁺(Z³)₃, wherein z is an integer selected from the range of from 1 to 20, and Z³ is a C1 to C5 alkyl group; and
   tripeptide Z¹-Proline-Z², wherein Z¹ and Z² are independently from each other an amino acid selected from the group consisting of alanine, glycine, valine, leucine, isoleucine and phenyl alanine, wherein the C terminus of Z² is preferably blocked, more preferably amidated.
R⁶, if present, is preferably a substituted or unsubstituted phenylene ring, wherein the one or more substituents are selected from hydrogen atom, halogen atom and functional group. In case R⁶ is a phenylene group, then R⁷ or, if R⁷ is absent the —C≡CH, is bound to the phenylene in ortho, meta or para position, preferably in para position, relative to the bond to the adjacent C atom of CR²R³. If R⁶ is absent, then the adjacent C atom of CR²R³ is directly bound to R⁷, if both R⁶ and R⁷ are absent, then the adjacent C atom of CR²R³ is directly bound to the —C≡CH. If R⁶ is a substituted or unsubstituted C6 to C10 arylene, then R⁷ or, if R⁷ is absent the —C≡CH, is bound to free position on the C6 to C10 arylene.

In these embodiments where the (poly)label has a structure of formula (Ia), it is preferred that the Z group(s), especially when the analyte of interest is a polypeptide, is/are not directly bound to the polypeptide's C- and/or N-terminus, but are rather always bound via Y, Y¹ respectively and X, as well with an optional Q linker.

Here and in the following, structures of Y or Y¹, which include a triazole ring, are preferably formed by the use of a specific amino acid in the synthesis of the (poly)label, which carries either a alkine group or an azide group in its side chain. Preferably, a precursor of the (poly)label comprises the alkine or azide in the following forms (IVa), (IVb):

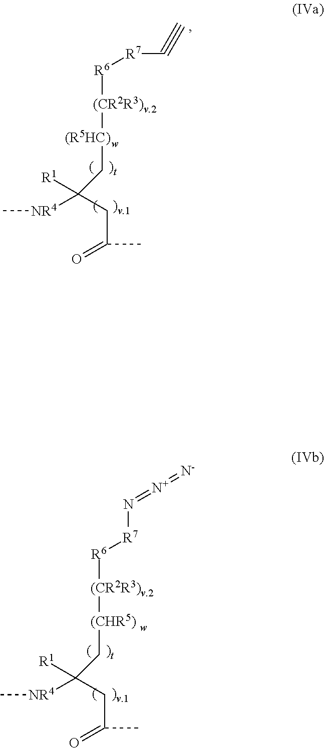

wherein
t is zero or 1;
v.1 is zero or an integer from the range of 1 to 4;
v.2 is an integer selected from the range of 1 to 10;
R¹ is a hydrogen atom or a C1-C5 straight or branched alkyl group;
R², R³ are independently from each other and independently for each of the v.2 units selected from hydrogen atom and C1-C5 straight or branched alkyl group;
R⁴ is a hydrogen atom or a C1-C5 straight or branched alkyl group;
R⁶ is absent or a —C(═O) NH— group or a C(═)O— group or a substituted or unsubstituted C6 to C10 arylene, wherein the one or more substituents are selected from hydrogen atom, halogen atom and functional group;
R⁷ is absent or selected from the group consisting of branched or unbranched C1-C5 alkylene, —O—C1 to C5 alkylene, wherein the C1 to C5 alkylene is branched or unbranched, and —S—C1 to C5 alkylene, wherein the C1 to C5 alkylene is branched or unbranched; or R⁴ and R⁵ together form a five or six membered heteroalkyl ring, which includes the nitrogen atom of NR⁴ as part of the ring structure. The dotted line at the NR⁴ or at the C(═O) for each (Iva), (IVb) represents the bond to the next unit.

Also for the precursor comprising (IVa) or (IVb) applies for R⁶, if present, is preferably a substituted or unsubstituted phenylene ring, wherein the one or more substituents are selected from hydrogen atom, halogen atom and functional group. In case R⁶ is a phenylene group, then R⁷ or, if R⁷ is absent the —C≡CH, is bound to the phenylene in ortho, meta or para position, preferably in para position, relative to the bond to the adjacent C atom of CR²R³. If R⁶ is absent, then the adjacent C atom of CR²R³ is directly bound to R⁷, if both R⁶ and R⁷ are absent, then the adjacent C atom of CR²R³ is directly bound to the —C≡CH. If Re is a substituted or unsubstituted C6 to C10 arylene, then R⁷ or, if R⁷ is absent the —C≡CH, is bound to free position on the C6 to C10 arylene.

For preparation of the (poly)label, the alkine or azide is then coupled via “click chemistry” with a corresponding azide or alkine, which carries Z, to form the (poly)label. In alternative embodiments, the coupling via “click chemistry” is done before the respective amino acid carrying the alkine or the azide is coupled via its amino and/or carboxylic group with others, i.e. in these embodiments, the structures (IVa) and (IVb) represent separate amino acids, wherein the dotted line at the NR⁴ for each (Iva), (IVb) represents a bond to a hydrogen atom or an activation group and the dotted line at the C(═O) for each (Iva), (IVb) represents the bond to a hydroxyl group or an activation group respectively.

A “click chemistry” means the, preferably Copper (I)-catalyzed, azide-alkyne cycloaddition (CuAAC). Reaction conditions, catalysts etc. for such a reaction are well known to the skilled person.

In some preferred embodiments of the use of a (poly)label for generating a quantifiable signal for an analyte of interest in mass spectrometry, the (poly)label has a structure of formula (Ia), wherein n is an integer selected from the range of from 1 to 20; X is a hydrogen atom; Q is absent,
Y is a

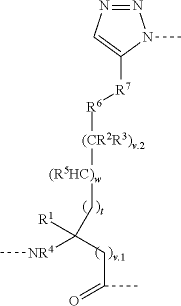

group, or a

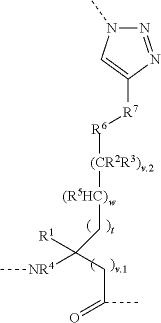

group,
or a

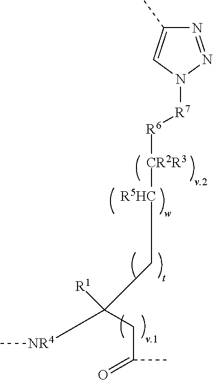

group, or a

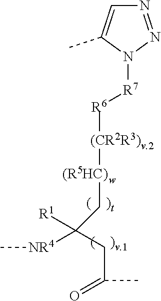

group;
wherein the dotted line at the NR⁴ represents the bond to Q or to the next [Y—Z] unit respectively, the dotted line at the C(═O) represents the bond to the next [Y—Z] unit or to Y¹ respectively and the dotted line at the N atom or the C atom in the triazole ring represent the bond to Z and t, v.1, v.2, w and R¹ to R⁵ have the same meaning as indicated above in embodiment 2;
Y¹ is a

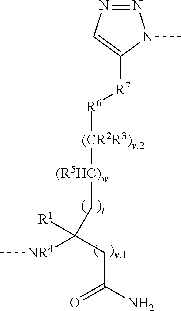

group, or a

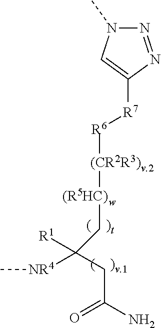

group,
   or a

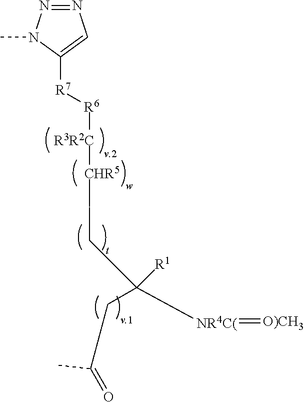

group, or a

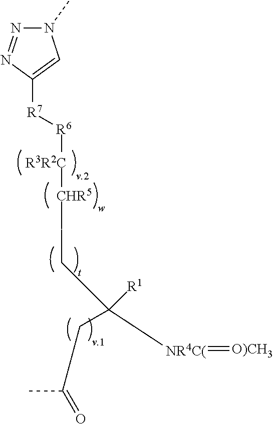

group,
   or a

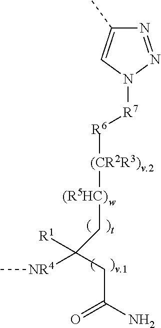

group, or a

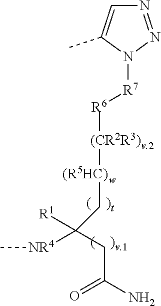

group,
or a

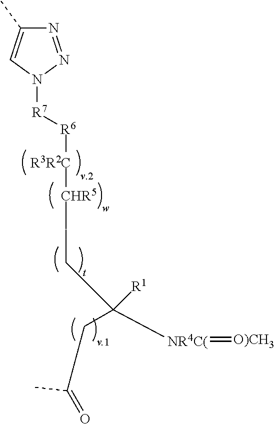

group, or a

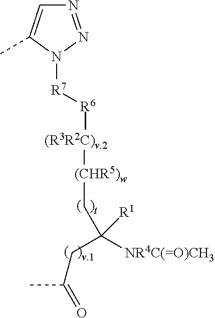

group;
wherein the dotted line at the NR⁴ or at the C(═O) represents the bond to the next [Y—Z] unit, the dotted line at the N atom or at the C atom in the triazole ring represent the bond to Z, and t, v.1, v.2, w and R¹ to R⁵ have the same meaning as indicated above in embodiment 2;
and
Z is a nucleoside with a nucleobase selected from the group consisting of adenine, cytosine, thymine, guanine and uracil.

In some preferred embodiments of the use of a (poly)label for generating a quantifiable signal for an analyte of interest in mass spectrometry, the (poly)label has a structure of formula (Ia), wherein n is an integer selected from the range of from 1 to 20; Q is a linker unit, X is a reactive group;
Y is a

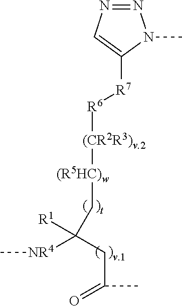

group, or a

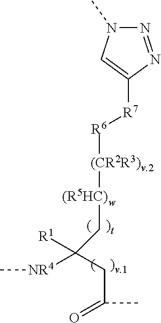

group,
   or a

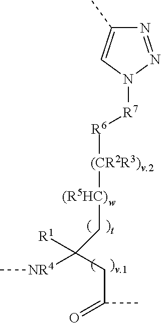

group, or a

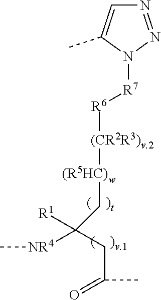

group,
wherein the dotted line at the NR⁴ represents the bond to Q or to the next [Y—Z] unit respectively, the dotted line at the C(═O) represents the bond to the next [Y—Z] unit or to Y¹ respectively and the dotted line at the N atom or at the C atom in the triazole ring represent the bond to Z and t, v.1, v.2, w and R¹ to R⁵ have the same meaning as indicated above in embodiment 2;
Y¹ is a

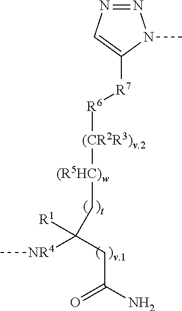

group, or a

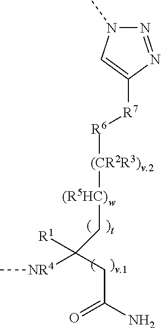

group,
   or a

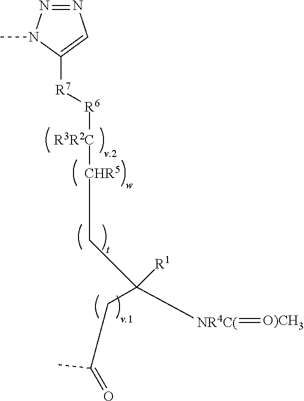

group, or a

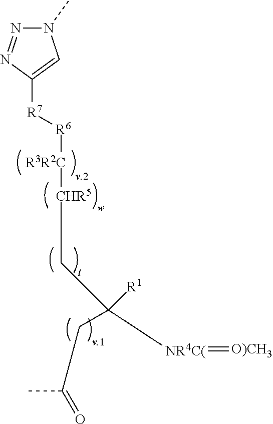

group,
or a

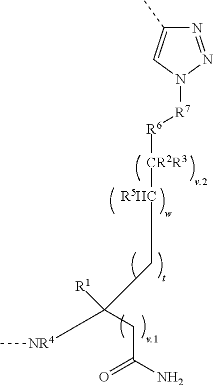

group, or a

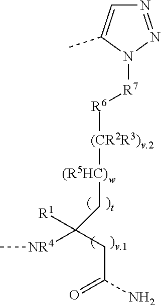

group,
or a

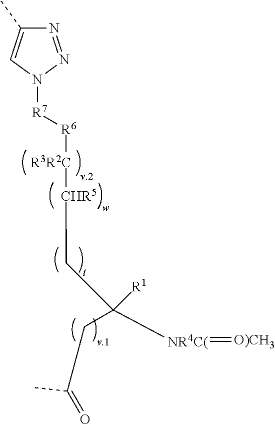

group, or a

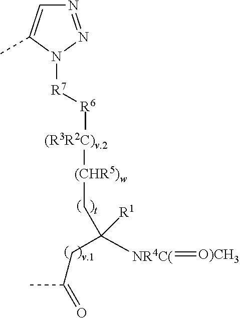

group;
wherein the dotted line at the NR⁴ or at the C(═O) represents the bond to the next [Y—Z] unit, the dotted line at the N atom or at the C atom in the triazole ring represent the bond to Z, and t, v.1, v.2, w and R¹ to R⁵ have the same meaning as indicated above in embodiment 2; and
Z is a carbamate group —O—C(═O)—NH—(CH₂)_{z}—N⁺(Z³)₃ or —NH—C(═O)—O—(CH₂)_{z}—N⁺(Z³)₃, wherein z is an integer selected from the range of from 1 to 10, and Z³ is a C1 to C5 alkyl group.

In some preferred embodiments of the use of a (poly)label for generating a quantifiable signal for an analyte of interest in mass spectrometry, the (poly)label has a structure of formula (Ia), wherein n is an integer selected from the range of from 1 to 20; Q is absent or is a linker unit; X is a hydrogen atom or a reactive group;
Y is a

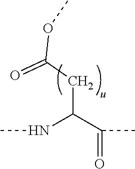

group, wherein the dotted line at the oxygen atom indicates the bond to Z and u is one or two, the dotted line at the NH represents the bond to Q or to the next [Y—Z] unit respectively, the dotted line at the C(═O) represents the bond to the next [Y—Z] unit or to Y¹ respectively;
Y¹ is a

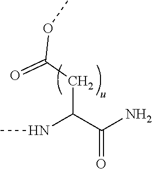

group, or a

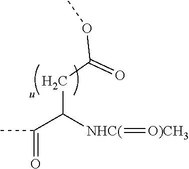

group,
   wherein the dotted line at the oxygen atom indicates the bond to Z and u is one or two, and the dotted line at the NH or at the C(═O) represents the bond to the next [Y—Z] unit; and
Z is a tripeptide Z¹-Proline-Z², wherein Z¹ and Z² are independently from each other an amino acid selected from the group consisting of alanine, glycine, valine, leucine, iso-leucine, and phenyl alanine, wherein the C terminus of Z² is preferably blocked, more preferably amidated.

In some preferred embodiments of the use of a (poly)label for generating a quantifiable signal for an analyte of interest in mass spectrometry,
Y is a

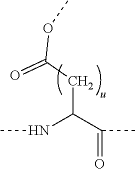

group,
   wherein the dotted line at the oxygen atom indicates the bond to Z and u is either one or two, the dotted line at the NH represents the bond to Q or to the next [Y—Z] unit respectively, the dotted line at the C(═O) represents the bond to the next [Y—Z] unit or to Y¹ respectively, or
   a

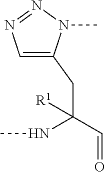

group, or a

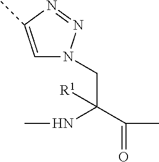

group,
wherein the dotted line at the N atom or at the C atom in the triazole ring represent the bond to Z and R¹ is a hydrogen atom or a methyl group, the dotted line at the NH for each group represents the bond to Q, to the next [Y—Z] unit or to Y¹, the dotted line at the C(═O) for each group represents the bond to the next [Y—Z] unit, to Y¹ or to Q, and u is either one or two.

In some preferred embodiments of the use of a (poly)label for generating a quantifiable signal for an analyte of interest in mass spectrometry,
Y¹ is a

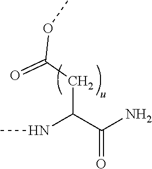

group, or a

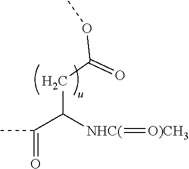

group,
   wherein the dotted line at the oxygen atom indicates the bond to Z and u is one or two, and the dotted line at the NH or at the C(═O) represents the bond to the next [Y—Z] unit, or
   a

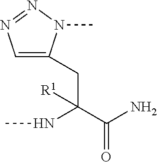

group, or a

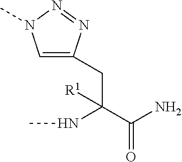

group,
or a

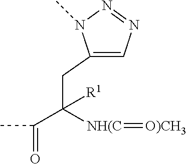

group, or a

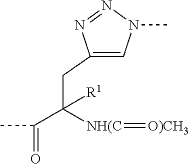

group,
or a

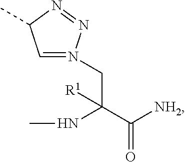

or a

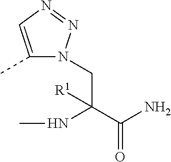

group,
or a

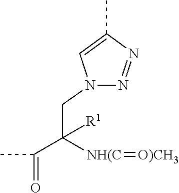

group, or a

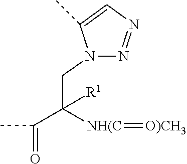

group,
wherein the dotted line at the NH or at the C(═O) represents the bond to the next [Y—Z] unit, the dotted line at the N atom or at the C atom in the triazole ring represent the bond to Z, and R¹ is a methyl group.

In some preferred embodiments of the use of a (poly)label for generating a quantifiable signal for an analyte of interest in mass spectrometry, in the (poly)label structure of formula (I) n and m are both zero, Q is absent and X is a hydrogen atom, the (poly)label having a structure of formula (Ib)

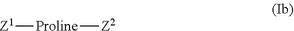

wherein Z¹ and Z² are independently from each other an amino acid selected from the group consisting of alanine, glycine, valine, leucine, iso-leucine, and phenyl alanine, wherein the C terminus of Z² is preferably blocked, more preferably amidated.

In some preferred embodiments of the use of a (poly)label for generating a quantifiable signal for an analyte of interest in mass spectrometry, Z¹ and Z² are independently from each other selected from alanine and glycine, wherein preferably Z¹ is alanine and Z² is glycine.

In these embodiments where the (poly)label has a structure of formula (Ib), it is preferred that the Z group(s), especially when the analyte of interest is a polypeptide, is/are directly bound to the polypeptide's C- and/or N-terminus.

In some preferred embodiments of the use of a (poly)label for generating a quantifiable signal for an analyte of interest in mass spectrometry, n is an integer selected from the range of from 1 to 10, preferably from the range of from 2 to 8.

In some preferred embodiments of the use of a (poly)label for generating a quantifiable signal for an analyte of interest in mass spectrometry, R¹ of Y of each of the n [Y—Z] units and R¹ of Y¹ are each a hydrogen atom.

In some preferred embodiments of the use of a (poly)label for generating a quantifiable signal for an analyte of interest in mass spectrometry, R¹ of Y of each of the n [Y—Z] units and R¹ of Y¹ are each a methyl group.

In some preferred embodiments of the use of a (poly)label for generating a quantifiable signal for an analyte of interest in mass spectrometry, for Z being a nucleoside, said nucleoside has the structure (IIa), (IIb), or (IIc):

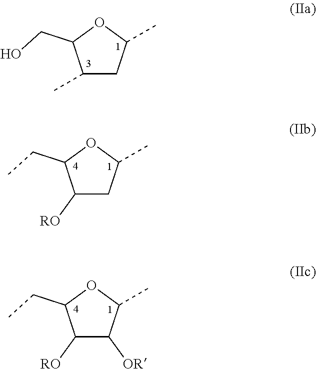

wherein the dotted line at position 1 of the five membered cycle represents the bond to the nucleobase and the dotted line at position 3 of the five membered cycle or at the CH₂ unit at position 4 of the five membered cycle represents the bond to Y and Y¹ respectively, preferably to the N atom in the triazole ring of Y and Y¹ respectively; and R, R′ is/are independently a hydrogen atom or a —CH₂—P(═O)(OH)₂ group.

In some preferred embodiments of the use of a (poly)label for generating a quantifiable signal for an analyte of interest in mass spectrometry, the nucleobase of the nucleoside Z is adenine.

In some preferred embodiments of the use of a (poly)label for generating a quantifiable signal for an analyte of interest in mass spectrometry, Z is carbamate group —O—C(═O)—NH—(CH₂)_{z}—N⁺(CH₃)₃ or —NH—C(═O)—O—(CH₂)_{z}—N⁺(Z³)₃, wherein z is an integer selected from the range of from 1 to 10, preferably selected from the range of from 2 to 5, more preferably z is 2.

In some preferred embodiments of the use of a (poly)label for generating a quantifiable signal for an analyte of interest in mass spectrometry, Q is a linker, which comprises a (C1-C5 alkylene-O—)ᵣ unit with r being an integer in the range of from 1 to 10 or a C1 to C20 alkanediyl unit or a C1 to C20 alkanediyl-heteroaryl unit or a (C1-C5 alkanediyl)-O—(C1-C5 alkanediyl) unit or a C2 to C9 alkanediyl-C(═O) unit or a C2 to C9 alkanediyl-NH unit, preferably a C4 to C7 alkanediyl-C(═O) unit or a C4 to C7 alkanediyl-NH unit, more preferably a hexylene-C(═O) unit or a hexylene-NH unit,

and
a [NH—C1 to C5 alkanediyl-C(═O)]ₓ unit or a [C(═O)—C1 to C5 alkanediyl-NH]ₓ unit, wherein x is an integer selected from the range of from 1 to 20, preferably from the range o from 2 to 8, more preferably from the range of from 3 to 7, more preferably from the range of from 4 to 6; and/or wherein the C1 to C5 alkanediyl is preferably a C2 to C4 alkanediyl, more preferably ethylene.

In some preferred embodiments of the use of a (poly)label for generating a quantifiable signal for an analyte of interest in mass spectrometry, X is a reactive group selected from the group consisting of isothiocyanate group, isocyanate group, acyl azide group, sulfonyl chloride group, aldehyde group, glyoxal group, epoxide group, oxirane group, carbonate group, aryl halide group, imidoester group, carbodiimide group, anhydride group, fluorophenyl ester group, carboxyl group,

HATU ester group, HBTU ester group and NHS ester group and is preferably a NHS ester group.

### 2^{nd} Aspect—Process for Creating a Quantifiable Signal for an Analyte of Interest

In a second aspect, the invention is related to a process for modifying an analyte of interest for obtaining an increased intensity signal in mass spectrometry, the process comprising the steps:
(a) providing at least one (poly)label having a reactive group of the structure (I)

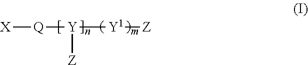

wherein:
m is zero or 1;
n is zero or an integer selected from the range of from 1 to 20;
Q is absent or a linker unit;
X is a reactive group or, if Q is absent, a hydrogen atom;
   Y is a linker unit based on an amino acid having a side chain suitable for coupling to Z;
   Y¹ is a linker unit based on an amino acid having a side chain suitable for coupling to Z, wherein the amino acid has a blocked carboxylic group or a blocked amino group;
Z is a moiety selected from the group consisting of
   nucleoside comprising a nucleobase selected from the group consisting of adenine, cytosine, thymine, guanine and uracil;
   tripeptide of structure Z¹-Proline-Z², wherein Z¹ and Z² are independently from each other an amino acid selected from the group consisting of alanine, glycine, valine, leucine, iso-leucine and phenyl alanine; and
   carbamate group —O—C(═O)—NH—(CH₂)_{z}—N⁺(Z³)₃ or —NH—C(═O)—O—(CH₂)_{z}—N⁺(Z³)₃, wherein z is an integer selected from the range of from 1 to 10, and Z³ is a C1 to C5 alkyl group.
(b) providing a analyte of interest, which is selected from the group consisting of polypeptide, and small molecule, and which is preferably a polypeptide, which has a free amino group and/or a free carboxyl group, wherein a free carboxyl group if present is optionally activated;
(c) reacting the at least one (poly)label having a reactive group with the analyte of interest, thereby obtaining a reaction product, wherein the at least one (poly)label is covalently bound to the analyte of interest.

All details, embodiments and preferred embodiments described above in the section related to the first aspect apply also for the second aspect of the invention.

In some preferred embodiments of the process for modifying an analyte of interest for obtaining an increased intensity signal in mass spectrometry, the analyte of interest is a small molecule, which is an organic compound having a molecular weight of ≤1000 daltons, wherein the small molecule is preferably a drug.

In some preferred embodiments of the process for modifying an analyte of interest for obtaining an increased intensity signal in mass spectrometry, the analyte of interest is a polypeptide which has a free amino group and/or a free carboxyl group, wherein the reaction product obtained in (c) is a compound having the general structure (III)

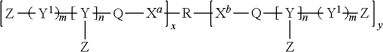

wherein Q, Y, Y¹, Z, m, and n have the meanings as defined above with respect to the first aspect of the invention, X^{a}, X^{b} are each a remainder of a group X as defined above with respect to the first aspect of the invention after having formed a, preferably covalent, bond with a corresponding functional group of the polypeptide, y and y are each zero or 1 with the condition that at least one of x, y is 1, and wherein R is the remainder of the polypeptide.

As indicated above in the section related to the first aspect, a “polypeptide” is a peptide wherein in the range of from 2 to 100 amino acids are bound by amide bonds. When R is the remainder of a polypeptide, then “remainder” means that the carboxyl group at the C terminus and/or the amino group at the N terminus of the peptide had formed an amide bond with a reactive group of the (poly)label. If x is zero, then the polypeptide's N terminus is still a —NH₂ group. If x is 1, then there is a remainder of a —NH₂ group such as —NH— at the polypeptide's N terminus. If y is zero, then the polypeptide's C terminus is still a COOH group. If y is 1, then there is a remainder of a COOH group such as —C(═O)— at the polypeptide's C terminus.

### 3^{rd} Aspect—Method for Determining an Analyte of Interest by Mass Spectrometry

A third aspect of the present invention is directed to a method for determining an analyte of interest by mass spectrometry, the method comprising:
(i) providing a reaction product of the analyte of interest, wherein the reaction product is based on a (poly)label having structure element (I)

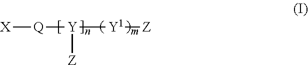

covalently bound to the analyte of interest, wherein Q, X, Y, Y¹, Y²Z, m and n have the meaning as defined in the sections related to the first aspect and the second aspect of the invention as described above;
(ii) subjecting the reaction product provided in (i) to mass spectrometry;
(iii) determining the intensity of a fragment corresponding to the MH⁺¹ peak of (unsubstituted) Z or of a water-deprived product thereof in the mass spectrum.

All details, embodiments and preferred embodiments described above in the sections related to the first and second aspect apply also for the third aspect of the invention.

### 4^{th} Aspect—Polylabel

A fourth aspect of the invention relates to a (poly)label having structure of formula (I)

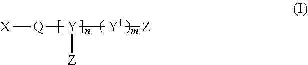

wherein Q, X, Y, Y¹, Y², Z, n and m have the meaning as defined in the section related to the first aspect of the invention above.

All details, embodiments and preferred embodiments described above in the sections related to the first, second and third aspect apply also for the fourth aspect of the invention.

### 5^{th} Aspect—Reaction Product

In a fifth aspect, the invention is directed to a reaction product comprising a polypeptide and a (poly)label having the general structure (III)

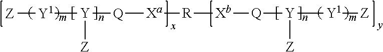

wherein Q, X, Y, Y¹, Y², Z, n and m have the meaning as defined in the sections related to the first, second, third and/or fourth aspect above and the indices x, y are either zero or 1 with the condition that at least one of x, y is 1, and wherein R is the remainder of the polypeptide.

All details, embodiments and preferred embodiments described above in the sections related to the first, second, third and fourth aspect apply also for the fifth aspect of the invention.

The present invention is further illustrated by the following embodiments and combinations of embodiments as indicated by the respective dependencies and back-references. In particular, it is noted that in each instance where a range of embodiments is mentioned, for example in the context of a term such as “The . . . of any of embodiments 1 to 4”, every embodiment in this range is meant to be explicitly disclosed for the skilled person, i.e. the wording of this term is to be understood by the skilled person as being synonymous to “The . . . of any of embodiments 1, 2, 3, and 4”.
1. Use of a (poly)label for generating a quantifiable signal for an analyte of interest in mass spectrometry, wherein the (poly)label has the structure (I)

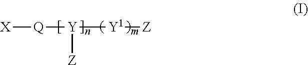

wherein
m is zero or 1;
n is zero or an integer selected from the range of from 1 to 20;
Q is absent or a linker unit;
X is a reactive group or, if Q is absent, a hydrogen atom;
Y is a linker unit based on an amino acid having a side chain suitable for coupling to Z;
Y¹ is a linker unit based on an amino acid having a side chain suitable for coupling to Z, wherein the amino acid has a blocked carboxylic group or a blocked amino group;
Z is a moiety selected from the group consisting of
   nucleoside comprising a nucleobase selected from the group consisting of adenine, cytosine, thymine, guanine and uracil;
   tripeptide of structure Z¹-Proline-Z², wherein Z¹ and Z² are independently from each other an amino acid selected from the group consisting of alanine, glycine, valine, leucine, iso-leucine and phenyl alanine; and
   carbamate group —O—C(═O)—NH—(CH₂)_{z}—N⁺(Z³)₃ or —NH—C(═O)—O—(CH₂)_{z}—N⁺(Z³)₃, wherein z is an integer selected from the range of from 1 to 10, and Z³ is a C1 to C5 alkyl group.
2. The use of embodiment 1, wherein the (poly)label has a structure of formula (Ia):

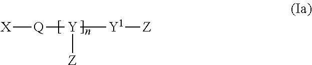

wherein
n is an integer selected from the range of from 1 to 20;
Q is absent or is a linker unit;
X is a reactive group;
Y is a

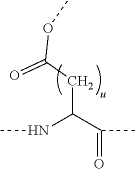

group, or a

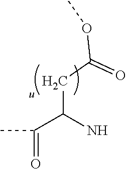

group, wherein the dotted line at the oxygen atom indicates the bond to Z and u is either one or two, or
   a

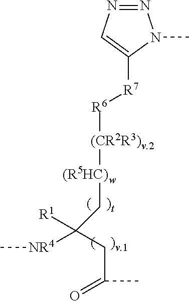

group, or a

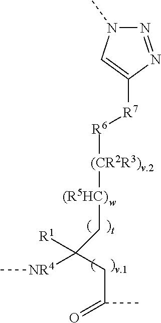

group,
or a

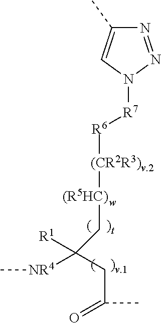

group, or a

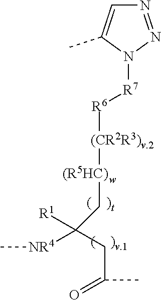

group, wherein the dotted line at the N atom or at the C atom in the triazole ring represent the bond to Z,
t is zero or 1;
v.1 is zero or an integer from the range of 1 to 4;
v.2 is an integer from the range of 1 to 10;
w is zero or 1;
R¹ is a hydrogen atom or a C1-C5 straight or branched alkyl group;
R², R³ are independently from each other and independently for each of the v.2 units selected from hydrogen atom and C1-C5 straight or branched alkyl group;
R⁴ is a hydrogen atom or a C1-C5 straight or branched alkyl group;
R⁵ is a hydrogen atom;
R⁶ is absent or a —C(═O)NH— group or a C(═)O— group or a substituted or unsubstituted C6 to C10 arylene, wherein the one or more substituents are selected from hydrogen atom, halogen atom and functional group;
R⁷ is absent or selected from the group consisting of branched or unbranched C1-C5 alkylene, —O—C1 to C5 alkylene, wherein the C1 to C5 alkylene is branched or unbranched, and —S—C1 to C5 alkylene, wherein the C1 to C5 alkylene is branched or unbranched;
or R⁴ and R⁵ together form a five or six membered heteroalkyl ring, which includes the nitrogen atom of NR⁴ as part of the ring structure;
wherein the dotted line at the NH or NR⁴ for each Y group represents the bond to Q, to the next [Y—Z] unit or to Y¹, the dotted line at the C(═O) for each group represents the bond to the next [Y—Z] unit, to Y¹ or to Q;
Y¹ is a

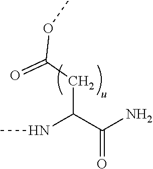

group or a

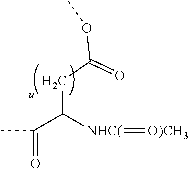

group, wherein the dotted line at the oxygen atom indicates the bond to Z and u is either one or two, or
   a

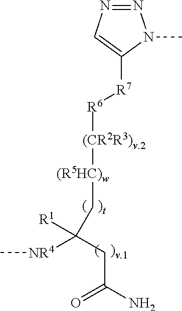

group, or a

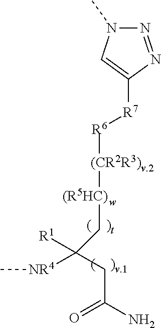

group,
or a

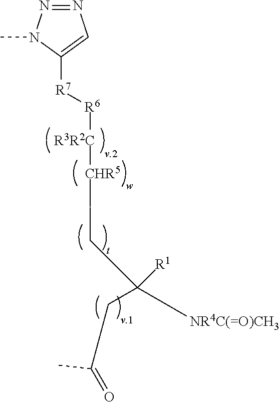

group, or a

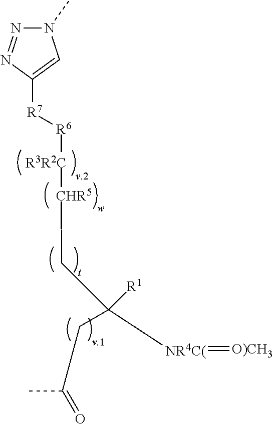

group,
or a

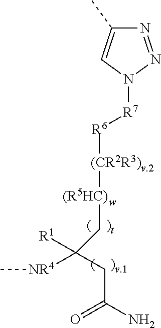

group, or a

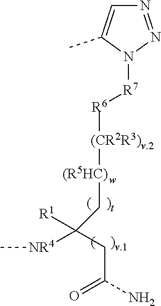

group,
or a

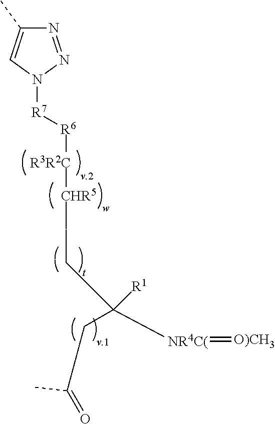

group, or a

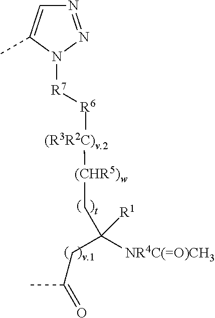

group,
wherein the dotted line at the N atom or at the C atom in the triazole ring represent the bond to Z, and t, v.1, v.2, w and R¹ to R⁵ have the same meaning as above for Y;
wherein the dotted line at the NH or NR⁴ or at the C(═O) for each Y¹ group represents the bond to the next [Y—Z] unit;
Z is a moiety selected from the group consisting of
   nucleoside with a nucleobase selected from the group consisting of adenine, cytosine, thymine, guanine and uracil; and
   carbamate group —O—C(═O)—NH—(CH₂)_{z}—N⁺(Z³)₃, or —NH—C(═O)—O—(CH₂)_{z}—N⁺(Z³)₃, wherein z is an integer selected from the range of from 1 to 20, and Z³ is a C1 to C5 alkyl group; and
   tripeptide Z¹-Proline-Z², wherein Z¹ and Z² are independently from each other an amino acid selected from the group consisting of alanine, glycine, valine, leucine, iso-leucine and phenyl alanine, wherein the C terminus of Z² is preferably blocked, more preferably amidated.
3. The use of embodiment 1 or 2, wherein the (poly)label has a structure of formula (Ia), wherein n is an integer selected from the range of from 1 to 20; X is a hydrogen atom; Q is absent,
   Y is a

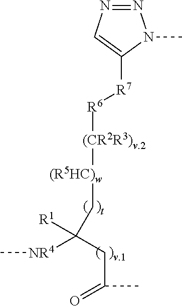

group, or a

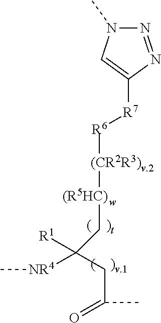

group,
   or a

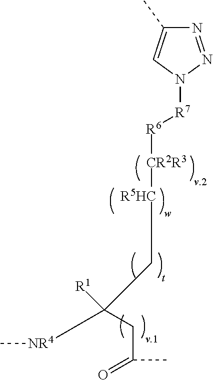

group, or a

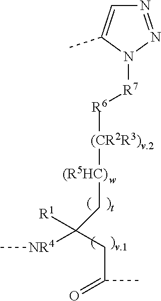

group;
wherein the dotted line at the NR⁴ represents the bond to Q or to the next [Y—Z] unit respectively, the dotted line at the C(═O) represents the bond to the next [Y—Z] unit or to Y¹ respectively and the dotted line at the N atom or the C atom in the triazole ring represent the bond to Z and t, v.1, v.2, w and R¹ to R⁵ have the same meaning as indicated above in embodiment 2;
Y¹ is a

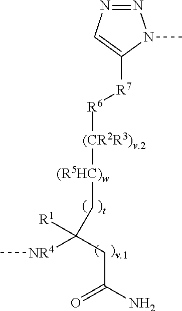

group, or a

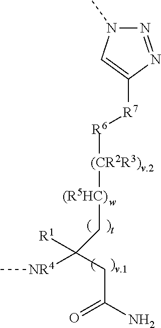

group,
   or a

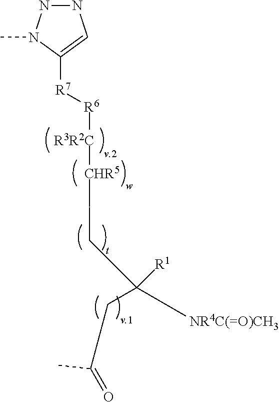

group, or a

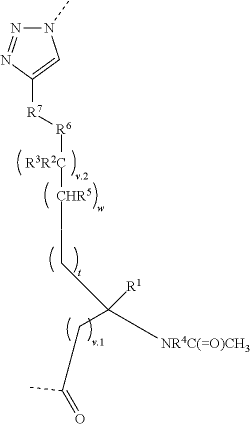

group,
or a

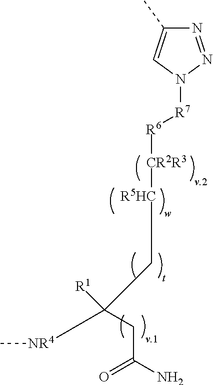

group, or a

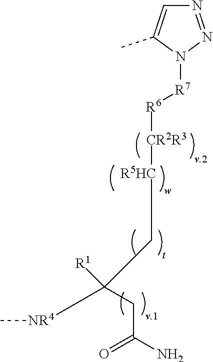

group,
or a

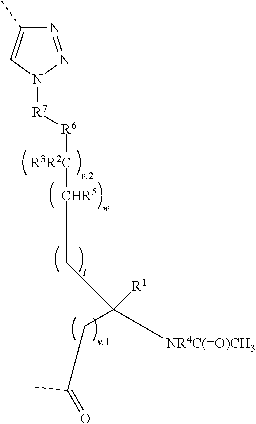

group, or a

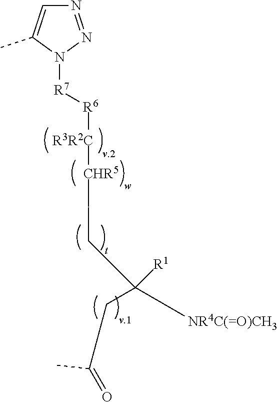

group;
wherein the dotted line at the NR⁴ or at the C(═O) represents the bond to the next [Y—Z] unit, the dotted line at the N atom or at the C atom in the triazole ring represent the bond to Z, and t, v.1, v.2, w and R¹ to R⁵ have the same meaning as indicated above in embodiment 2;
and
Z is a nucleoside with a nucleobase selected from the group consisting of adenine, cytosine, thymine, guanine and uracil.
4. The use of embodiment 1 or 2, wherein the (poly)label has a structure of formula (Ia), wherein n is an integer selected from the range of from 1 to 20; Q is a linker unit, X is a reactive group;
   Y is a

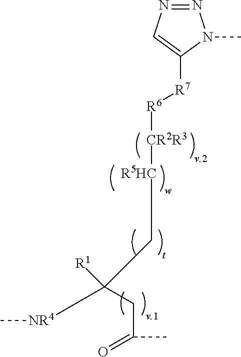

group, or a

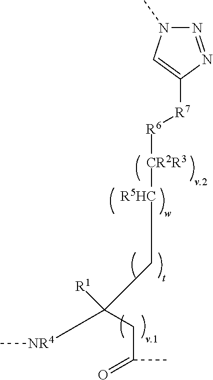

group,
or a

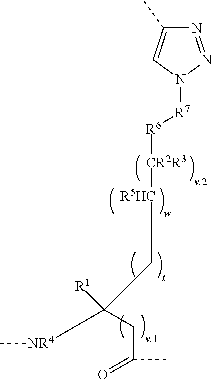

group, or a

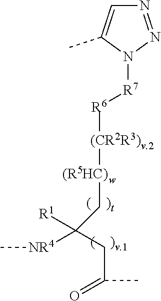

group,
wherein the dotted line at the NR⁴ represents the bond to Q or to the next [Y—Z] unit respectively, the dotted line at the C(═O) represents the bond to the next [Y—Z] unit or to Y¹ respectively and the dotted line at the N atom or at the C atom in the triazole ring represent the bond to Z and t, v.1, v.2, w and R¹ to R⁵ have the same meaning as indicated above in embodiment 2;
Y¹ is a

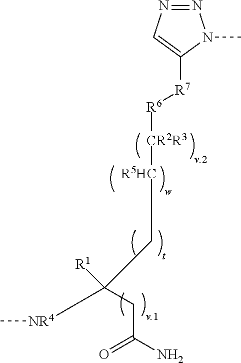

group, or a

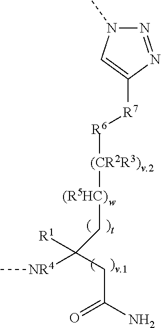

group,
   or a

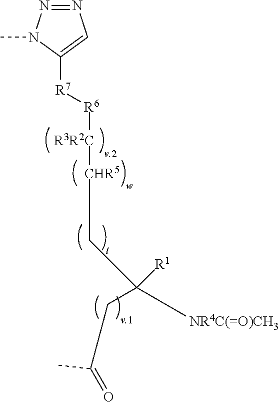

group, or a

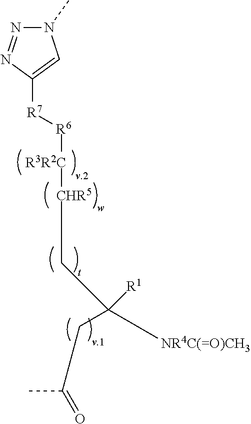

group,
or a

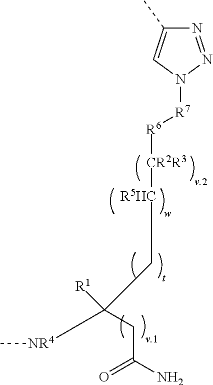

group, or a

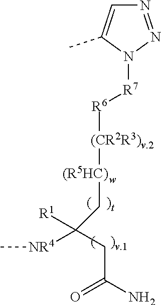

group,
or a

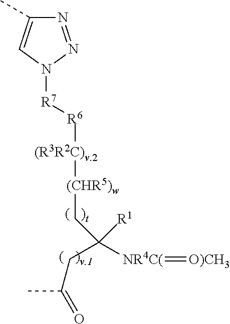

group, or a

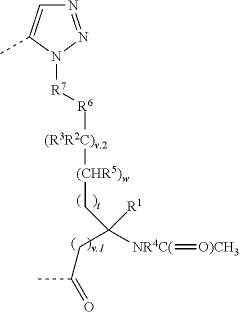

group;
wherein the dotted line at the NR⁴ or at the C(═O) represents the bond to the next [Y—Z] unit, the dotted line at the N atom or at the C atom in the triazole ring represent the bond to Z, and t, v.1, v.2, w and R¹ to R⁵ have the same meaning as indicated above in embodiment 2;
and
Z is a carbamate group —O—C(═O)—NH—(CH₂)_{z}—N⁺(Z³)₃ or —NH—C(═O)—O—(CH₂)_{z}—N⁺(Z³)₃, wherein z is an integer selected from the range of from 1 to 10, and Z³ is a C1 to C5 alkyl group.
5. The use of embodiment 1 or 2, wherein the (poly)label has a structure of formula (Ia), wherein n is an integer selected from the range of from 1 to 20; Q is absent or is a linker unit; X is a hydrogen atom or a reactive group;
   Y is a

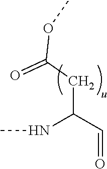

group, wherein the dotted line at the oxygen atom indicates the bond to Z and u is one or two, the dotted line at the NH represents the bond to Q or to the next [Y—Z] unit respectively, the dotted line at the C(═O) represents the bond to the next [Y—Z] unit or to Y¹ respectively;
Y¹ is a

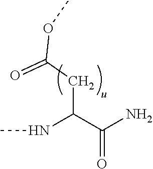

group, or a

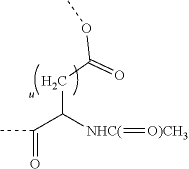

group,
   wherein the dotted line at the oxygen atom indicates the bond to Z and u is one or two, and the dotted line at the NH or at the C(═O) represents the bond to the next [Y—Z] unit;
and
Z is a tripeptide Z¹-Proline-Z², wherein Z¹ and Z² are independently from each other an amino acid selected from the group consisting of alanine, glycine, valine, leucine, isoleucine, and phenyl alanine, wherein the C terminus of Z² is preferably blocked, more preferably amidated.
6. The use of any one of embodiments 2 to 5, wherein
   Y is a

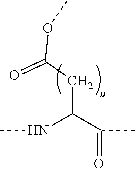

group,
   wherein the dotted line at the oxygen atom indicates the bond to Z and u is either one or two, the dotted line at the NH represents the bond to Q or to the next [Y—Z] unit respectively, the dotted line at the C(═O) represents the bond to the next [Y—Z] unit or to Y¹ respectively, or
   a

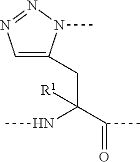

group, or a

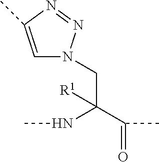

group,
wherein the dotted line at the N atom or at the C atom in the triazole ring represent the bond to Z and R¹ is a hydrogen atom or a methyl group, the dotted line at the NH for each group represents the bond to Q, to the next [Y—Z] unit or to Y¹, the dotted line at the C(═O) for each group represents the bond to the next [Y—Z] unit, to Y¹ or to Q, and u is either one or two.
7. The use of any one of embodiments 2 to 6, wherein
   Y¹ is a

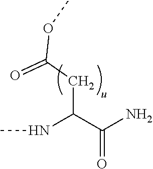

group, or a

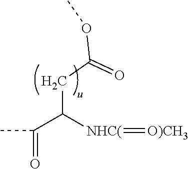

group,
   wherein the dotted line at the oxygen atom indicates the bond to Z and u is one or two, and the dotted line at the NH or at the C(═O) represents the bond to the next [Y—Z] unit, or
   a

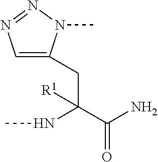

group, or a

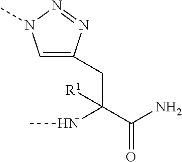

group,
or a

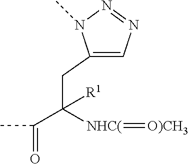

group, or a

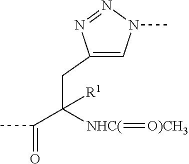

group,
or a

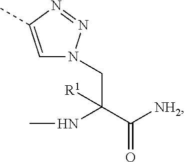

or a

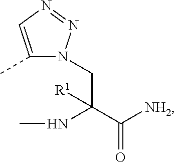

group,
or a

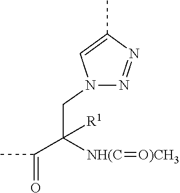

group, or a

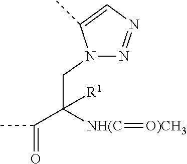

group,
wherein the dotted line at the NH or at the C(═O) represents the bond to the next [Y—Z] unit, the dotted line at the N atom or at the C atom in the triazole ring represent the bond to Z, and R¹ is a methyl group.
8. The use of embodiment 1 or 2, wherein in the (poly)label structure of formula (I) n and m are both zero, Q is absent and X is a hydrogen atom, the (poly)label having a structure of formula (Ib)

Z¹-Proline-Z² (Ib)
wherein Z¹ and Z² are independently from each other an amino acid selected from the group consisting of alanine, glycine, valine, leucine, iso-leucine, and phenyl alanine, wherein the C terminus of Z² is preferably blocked, more preferably amidated.
9. The use of any one of embodiments 1 to 8, wherein Z¹ and Z² are independently from each other selected from alanine and glycine, wherein preferably Z¹ is alanine and Z² is glycine.
10. The use of any one of embodiments 1 to 9, wherein n is an integer selected from the range of from 1 to 10, preferably from the range of from 2 to 8.
11. The use of any one of embodiments 1 to 10, wherein R¹ of Y of each of the n [Y—Z] units and R¹ of Y¹ are each a hydrogen atom.
12. The use of any one of embodiment 1 to 9, wherein R¹ of Y of each of the n [Y—Z] units and R¹ of Y¹ are each a methyl group.
13. The use of any one of embodiments 1 to 12, wherein for Z being a nucleoside, said nucleoside has the structure (IIa), (IIb), or (IIc):

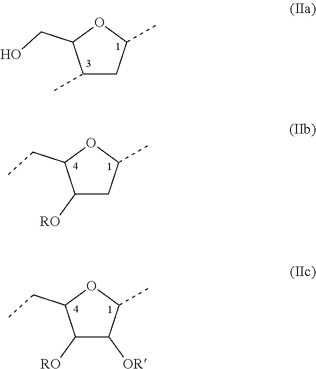

wherein the dotted line at position 1 of the five membered cycle represents the bond to the nucleobase and the dotted line at position 3 of the five membered cycle or at the CH₂ unit at position 4 of the five membered cycle represents the bond to Y and Y¹ respectively, preferably to the N atom in the triazole ring of Y and Y¹ respectively; and R, R′ is/are independently a hydrogen atom or a —CH₂—P(═O)(OH)₂ group.
14. The use of any one of embodiments 1 to 13, wherein the nucleobase of the nucleoside Z is adenine.
15. The use of any one of embodiments 1 to 14, wherein Z is carbamate group —O—C(═O)—NH—(CH₂)_{z}—N⁺(CH₃)₃ or —NH—C(═O)—O—(CH₂)_{z}—N⁺(Z³)₃, wherein z is an integer selected from the range of from 1 to 10, preferably selected from the range of from 2 to 5, more preferably z is 2.
16. The use of any one of embodiments 1 to 15, wherein Q is a linker, which comprises a (C1-C5 alkylene-O—) r unit with r being an integer in the range of from 1 to 10 or a C1 to C20 alkanediyl unit or a C1 to C20 alkanediyl-heteroaryl unit or a (C1-C5 alkanediyl)-O—(C1-C5 alkanediyl) unit or a C2 to C9 alkanediyl-C(═O) unit or a C2 to C9 alkanediyl-NH unit, preferably a C4 to C7 alkanediyl —C(═O) unit or a C4 to C7 alkanediyl-NH unit, more preferably a hexylene-C(═O) unit or a hexylene-NH unit,
and
a [NH—C1 to C5 alkanediyl-C(═O)]ₓ unit or a [C(═O)—C1 to C5 alkanediyl-NH]ₓ unit, wherein x is an integer selected from the range of from 1 to 20, preferably from the range o from 2 to 8, more preferably from the range of from 3 to 7, more preferably from the range of from 4 to 6; and/or wherein the C1 to C5 alkanediyl is preferably a C2 to C4 alkanediyl, more preferably ethylene.
17. The use of any one of embodiments 1 to 16, wherein X is a reactive group selected from the group consisting of isothiocyanate group, isocyanate group, acyl azide group, sulfonyl chloride group, aldehyde group, glyoxal group, epoxide group, oxirane group, carbonate group, aryl halide group, imidoester group, carbodiimide group, anhydride group, fluorophenyl ester group, carboxyl group, HATU ester group, HBTU ester group and NHS ester group and is preferably a NHS ester group.
18. A process for modifying an analyte of interest for obtaining an increased intensity signal in mass spectrometry, the process comprising the steps:
   (b) providing at least one (poly)label having a reactive group of the structure (I)

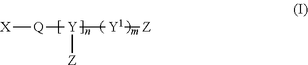

wherein:
m is zero or 1;
n is zero or an integer selected from the range of from 1 to 20;
Q is absent or a linker unit;
X is a reactive group or, if Q is absent, a hydrogen atom;
Y is a linker unit based on an amino acid having a side chain suitable for coupling to Z;
Y¹ is a linker unit based on an amino acid having a side chain suitable for coupling to Z, wherein the amino acid has a blocked carboxylic group or a blocked amino group;
Z is a moiety selected from the group consisting of
   nucleoside comprising a nucleobase selected from the group consisting of adenine, cytosine, thymine, guanine and uracil;
   tripeptide of structure Z¹-Proline-Z², wherein Z¹ and Z² are independently from each other an amino acid selected from the group consisting of alanine, glycine, valine, leucine, iso-leucine and phenyl alanine; and
   carbamate group-O—C(═O)—NH—(CH₂)_{z}—N⁺(Z³)₃ or —NH—C(═O)—O—(CH₂)_{z}N⁺(Z³)₃, wherein z is an integer selected from the range of from 1 to 10, and Z³ is a C1 to C5 alkyl group.
(b) providing a analyte of interest, which is selected from the group consisting of polypeptide, and small molecule, and which is preferably a polypeptide, which has a free amino group and/or a free carboxyl group, wherein a free carboxyl group if present is optionally activated;
(c) reacting the at least one (poly)label having a reactive group with the analyte of interest, thereby obtaining a reaction product, wherein the at least one (poly)label is covalently bound to the analyte of interest.
19. The process of embodiment 18, wherein the analyte of interest is a small molecule, which is an organic compound having a molecular weight of ≤1000 daltons, wherein the small molecule is preferably a drug.
20. The process of embodiment 18, wherein the analyte of interest is a polypeptide which has a free amino group and/or a free carboxyl group, wherein the reaction product obtained in (c) is a compound having the general structure (III)

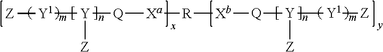

wherein Q, Y, Y¹, Z, m, and n have the meanings as defined in any one of embodiments 1 to 17, X^{a}, X^{b} are each a remainder of a group X as defined in any one of embodiments 1 to 17 after having formed a, preferably covalent, bond with a corresponding functional group of the polypeptide, y and y are each zero or 1 with the condition that at least one of x, y is 1, and wherein R is the remainder of the polypeptide.
21. A method for determining an analyte of interest by mass spectrometry, the method comprising:
   (ii) providing a reaction product of the analyte of interest, wherein the reaction product is based on a (poly)label having structure element (I)

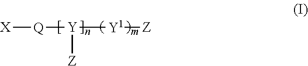

wherein
covalently bound to the analyte of interest, wherein Q, X, Y, Y¹, Y²Z, m and n have the meaning as defined in any one of the embodiments above;
(ii) subjecting the reaction product provided in (i) to mass spectrometry;
(iii) determining the intensity of a fragment corresponding to the MH⁺¹ peak of (unsubstituted) Z or of a water-deprived product thereof in the mass spectrum.
22. A (poly)label having structure of formula (I)

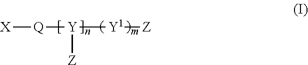

wherein Q, X, Y, Y¹, Y², Z, n and m have the meaning as defined in any one of the embodiments above.
23. A reaction product comprising a polypeptide and a (poly)label having the general structure (III)

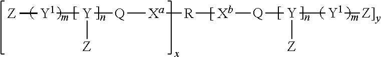

wherein Q, X, Y, Y¹, Y², Z, n and m have the meaning as defined in any one of the embodiments above and the indices x, y are either zero or 1 with the condition that at least one of x, y is 1, and wherein R is the remainder of the polypeptide.

The present invention is further illustrated by the following reference examples, comparative examples, and examples.

### EXAMPLES

### Reference Example 1: Dissolving Synthetic Peptides

Gravimetrically measured synthetic peptides were dissolved with 0.1% CH₂O₂, 5% C₂H₃N in H₂O at a concentration of 1 μM.

### Reference Example 2: Liquid Chromatography Tandem-Mass Spectrometry (LC-MS/MS)

### Tuning of MS Parameters

A triple quadrupole mass spectrometer was tuned for each respective synthetically (poly)labelled peptides by using direct infusion strategy, in which a T-junction was used to combine the flow from the syringe pump, delivering 1 μM (poly)labelled peptide solution at a flow rate of 5 μL/min and LC flow (295 μL/min).

### HPLC Parameters

Vanquish UHPLC Autosampler, Thermo Scientific
Vanquish UHPLC Pump, Thermo Scientific
Vanquish UHPLC Column Compartment, Thermo Scientific
Coupled to TSQ Quantiva, Thermo Scientific
Column: Acuity UPLC BEH C18 1.7 μM, 2.1×100 mm
Buffer A: 0.1% CH₂O₂ in H₂O
Buffer B: 0.1% CH₂O₂ in C₂H₃N

### Flow Gradient

| Time (min) | Flow (mL/min) | % B |
|---|---|---|
| 0.0 | 0.3 | 2.0 |
| 2.0 | 0.3 | 2.0 |
| 7.5 | 0.3 | 40.0 |
| 7.6 | 0.3 | 90.0 |
| 10.0 | 0.3 | 90.0 |
| 10.1 | 0.3 | 2.0 |
| 15.0 | 0.3 | 2.0 |

### Autosampler PARAMETERS:

Draw speed: 0.5 μl/s
Dispense speed: 5.0 μl/s

### Column Chamber:

Use temperature control: ticked
Temperature (° C.): 50.0

### MS Parameters

Ion source type: H-ESI
Spray Voltage: Static
Polarity: Positive
Ion transfer Tube Temp. (C): 275
Vaporizer Temp. (° C.): 325
Dwell Time (ms): 50
Use Calibrated RF Lens: ticked
Q1 Resolution (FWHM): 0.7
Q3 Resolution (FWHM): 0.7
CID Gas (mTorr): 2
Spray Voltage and Collision Energy (V) is analyte specific.

### Data Analysis

Data Analysis was performed with Chromatography Data System Software “Chromeleon 7” of Thermo Scientific (version 7.3.1 CDS)

Fragmentation efficiency=Product area under the curve/Precursor area under the curve*100

The quantifier ions based on the individual (poly)labels of formula (I) were as listed below in Table 1:

TABLE 1

| | | Elemental | |
|---|---|---|---|
| Z of formula (I) | Quantifier Moiety | Composition | MH⁺¹(Da) |
| Nucleoside with | adeninine | C5H6N5 | 136 |
| adenine as nucleo- | | | |
| base | | | |
| Carbamate group | Water deprived car- | C6H13N2O | 129 |
| | bamate group | | |
| Tripeptide APG | PG | C7H12N2O3 | 172 |

### Reference Example 3: Derivatization of Tryptic Peptides at the N Terminus

Tryptic Peptides with the following peptide sequences (sequences from N to C terminus) were derivatized:

Peptide 1
(SEQ ID No. 1)
ATNSQFLR
Peptide 2
(SEQ ID No. 2)
FSPDDSAGASALLR
Peptide 3
(SEQ ID No. 3)
VIFDANAPVAVR

Derivatization at the N terminus of a peptide was done with the following carbamate based (poly)label tags:

1)
DSS-UUUU*Prg*Prg**NH2
2)
DSS-UUUU*Prg*Prg*Prg*Prg**NH2

wherein “DSS” represents the remainder of disuccinimidylsuberate with a remaining NHS ester group, “U” represents the remainder of 3-aminopropanoic acid, “Prg*” represents the remainder of the (click chemistry) reaction product of propargylglycine and N₃⁺—(CH₂)₃—O—C(═O)—NH—(CH₂)₂—N⁺(CH₃)₃, wherein “Prg**NH2” at the end indicates that the terminal COOH group of the final propargylalanine is amidated. The structures of (poly)label tags (1) and (2) are shown below:

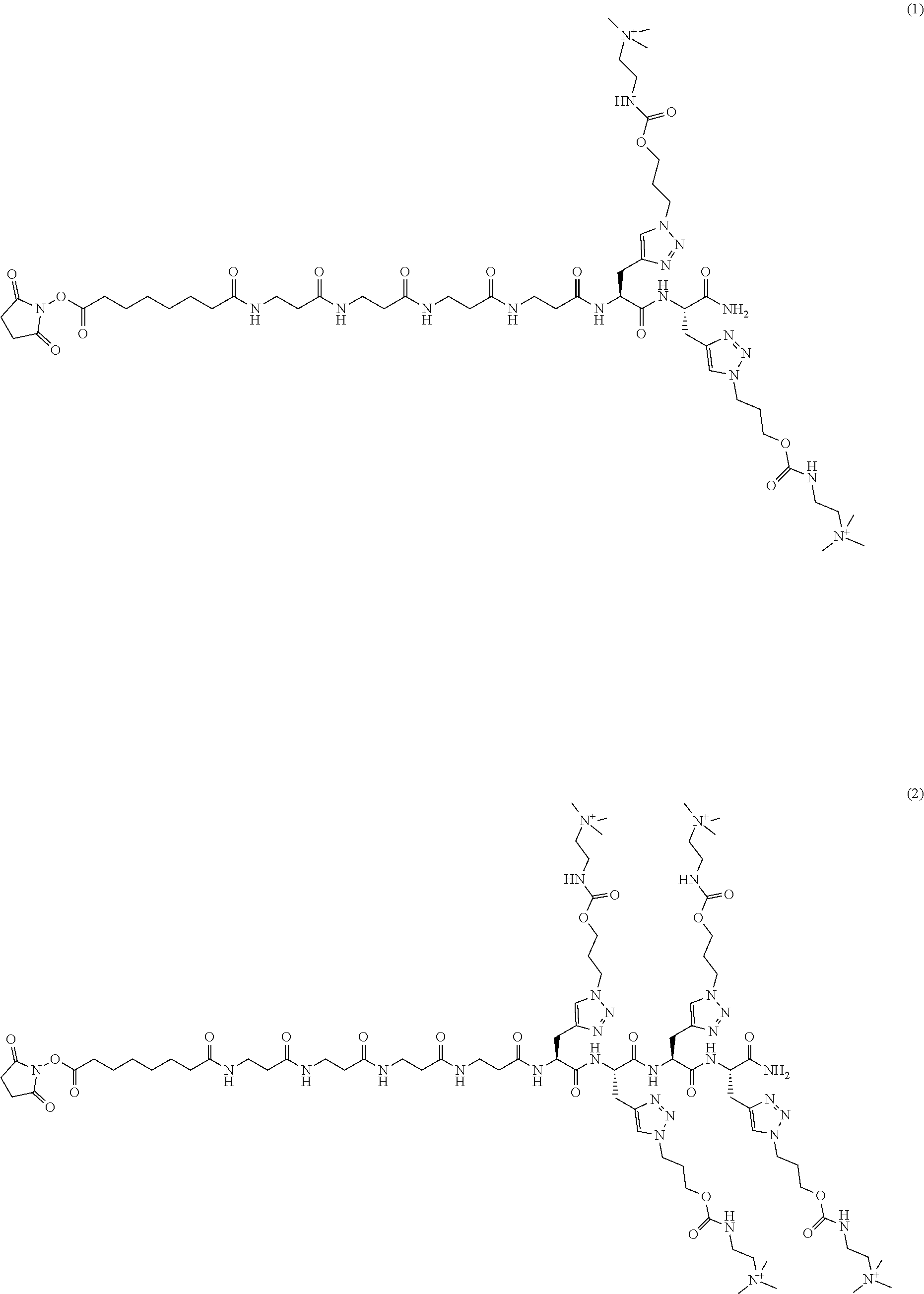 [SMILES: C[N+](C)(C)CCNC(=O)OCCCn1cc(C[C@H](NC(=O)[C@H](Cc2cn(CCCOC(=O)NCC[N+](C)(C)C)nn2)NC(=O)CCNC(=O)CCNC(=O)CCNC(=O)CCNC(=O)CCCCCCC(=O)ON2C(=O)CCC2=O)C(N)=O)nn1 ; C[N+](C)(C)CCNC(=O)OCCCn1cc(C[C@H](NC(=O)[C@H](Cc2cn(CCCOC(=O)NCC[N+](C)(C)C)nn2)NC(=O)[C@H](Cc2cn(CCCOC(=O)NCC[N+](C)(C)C)nn2)NC(=O)[C@H](Cc2cn(CCCOC(=O)NCC[N+](C)(C)C)nn2)NC(=O)CCNC(=O)CCNC(=O)CCNC(=O)CCNC(=O)CCCCCCC(=O)ON2C(=O)CCC2=O)C(N)=O)nn1]

### Synthesis of (Poly)Label Tags

### a) Synthesis of H-UUUU-PrgPrgNH2 or H-UUUUPrgPrgPrgPrgNH2

Peptides were synthesized by means of fluorenylmethyloxycarbonyl (Fmoc) solid phase peptide synthesis on a peptide synthesizer (e.g. from Protein Technologies, Inc). For amino acid couplings 5 equivalents of each amino acid derivative (Fmoc-propargyl-glycine and Fmoc-beta-alanine) were used. Amino acid derivatives were dissolved in dimethylformamide containing 1 equivalent of 1-Hydroxy-7-azabenzotriazol (HOAt). Peptides were synthesized on Tentagel R resin. Coupling reactions were carried out for 5 minutes in dimethylformamide with 5 equivalents HATU and 10 equivalents of N,N-Diisopropylethylamine relative to resin loading. The Fmoc-group was cleaved for 8 minutes after each synthesis step using 20% piperidine in dimethylformamide. Release of the peptide from the synthesis resin was achieved by incubation with 95% TFA, 2.5% triisopropylsilane and 2.5% water for 3 hours. The reaction solution was subsequently mixed cooled diisopropyl ether to precipitate the peptide. The precipitate was filtered, washed again with diisopropyl ether, dissolved in a small amount of aqueous acetic acid and lyophilized. The crude material obtained was purified by preparative RP-HPLC using a gradient of acetonitrile/water containing 0.1% trifluoroacetic acid. The identity of the purified material was checked by means of ion spray mass spectrometry.

### b) Attachment of Label Groups: Synthesis of H-UUUU*Prg*Prg**NH2 or H-UUUU*Prg*Prg*Prg*Prg**NH2

Alkyne-containing peptide prepared in (a) (1 equivalent) and azide-containing label (4.4 equivalents), such as N₃⁺—(CH₂)₃—O—C(═O)—NH—(CH₂)₂—N⁺(CH₃)₃, were dissolved in water/acetonitrile (1:1) and a solution of CuBr (1.5 equivalents, 0.1 M in acetonitrile), THPTA (1.5 equivalents, 0.1 M in water) and trimethylamine (3 equivalents) was added. The reaction mixture was kept under an atmosphere of argon and shaken (700 rpm) at 32° C. After 20 h a solution of EDTA (pH 8.0) was added and the mixture was shaken for 10 minutes at room temperature. Afterwards the solution was diluted with water (1:1), filtrated and purified by flash chromatography on a C-18 column. Product containing fractions were pooled and lyophilized. The identity of the product was verified by LC-MS and 1H-NMR.

### c) Attachment of DSS to (Poly)Label Peptides

To a solution of disuccinimidylsuberate (DSS) (1 equivalent) in dry DMF a peptide obtained in (b) (1,3 equivalents) dissolved in DMF and diisopropylamine (2 equivalents) was added and stirred for 2 hours at room temperature. The product was purified by preparative HPLC. The identity of the purified material was checked by means of ion spray mass spectrometry.

For derivatization at the N terminus of a peptide, 30 μM peptides were reacted with 150 μM of (poly)label tag (1) or (2) as obtained from (c) in 100 mM aqueous sodium bicarbonate at pH 8.0 for 3 hours at 37° C.

### Reference Example 4: Synthesis of Tryptic Peptides Having a (Poly)Label at the C Terminus

Peptide 1 (ATNSQFLR, SEQ ID No. 1) was derivatived with Prg^{##}NH2 [(poly)label tag (3)], wherein “Prg^{##}NH2” represents the remainder of a reaction product of progargylglycine coupled via its alkenyl group (via click chemistry) with a N³⁺—Z group, wherein Z is a nucleoside having adenine as base, and wherein the terminal COOH group of the propargylglycine is amidated. Peptide 1 (ATNSQFLR, SEQ ID No. 1) was also derivatived with Prg^{#}Prg^{##}NH2 [(poly)label tag (4)], wherein “Prg^{#}” represents the remainder of a reaction product of propargylglycine coupled via its alkenyl group (via click chemistry) with a N³⁺—Z group, wherein Z is a nucleoside having adenine as base, and wherein “Prg^{##}NH2” at the end indicates that the terminal COOH group of the final propargylglycine is amidated. The resulting structure is shown below:

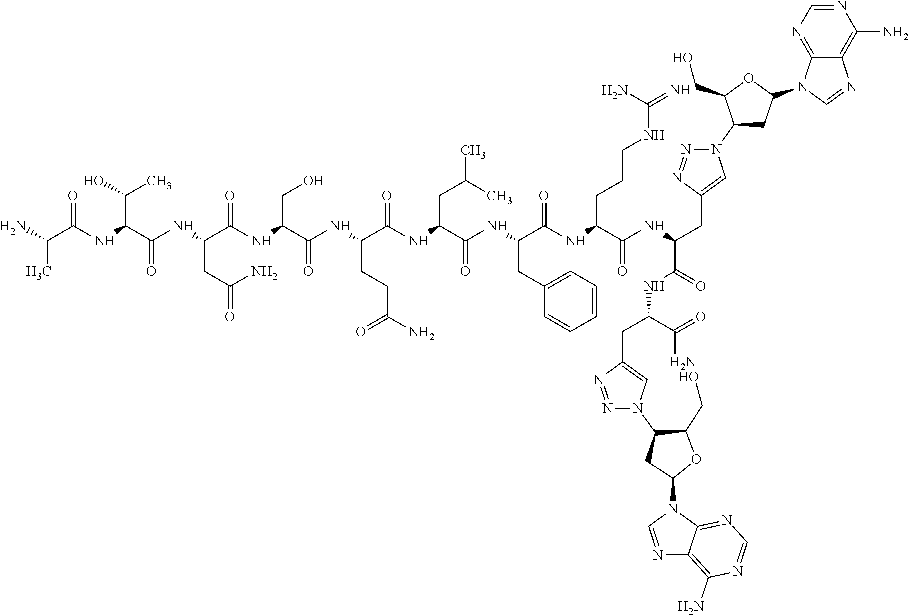

### Comparative Example 1: MS/MS Spectrum of a Peptide ATNSQLFR Without Any (poly)label

Selective fragmentation of peptide 1 ATNSQFLR (SEQ ID No. 1) was investigated by MS/MS. The respective MS/MS spectrum is shown in FIG. 1.

Under collusion induced dissociation (CID) conditions within the mass spectrometer, the peptide collided with inert gas and—as usual—broke apart at a weak bond, which was typically one of the amide bonds to generate peptide fragment ions (FIG. 1), resulting in a high number of fragment ions as shown in FIG. 1. This limited sensitive detection of the peptide because the signal was divided into many fragment ions as shown in FIG. 1.

### Example 1: Selective Fragmentation to Generate Quantifier Ion-MS/MS Spectrum of a Peptide Having an Adenine Label at the C Terminus

Peptide 1a comprising peptide 1 (ATNSQFLR, SEQ ID No. 1) having a single adenine containing poly label bound at the C terminus, prepared according to Reference Example 4, was investigated via MS/MS. The MS/MS spectrum is shown in FIG. 2. It was shown that, unlike a peptide without (poly)label (see Comparative Example 1, FIG. 1), the peptide-(polyl)abel construct with adenine containing (poly)label as label apart selectively broke apart to generate a high abundant quantifier ion (136 Da).

### Example 2: Selective Fragmentation to Generate Quantifier Ion-MS/MS Spectrum Of a Peptide Having an APG Label at the C Terminus

Peptide 1 ATNSQFLR (SEQ ID No. 1) having a blocked (acetylated) N terminus was derivatized with 1 APG-containing (poly)label at the C terminus, i.e. a glutamic acid carrying a APG group coupled to the free COOH group of the glutamic acid side chain by an amide bond and having the C-term of the final glutamic acid amidated was coupled to peptide 1's C terminus, was investigated via MS/MS. The MS/MS spectrum is shown in FIG. 3. It was shown that, unlike a peptide without (poly)label (see Comparative Example 1, FIG. 1), the peptide-(poly)label based on APG generated a high abundant “quantifier ion” (PG, Proline-Glycine, MH⁺¹=172 Da).

### Example 3: Selective Fragmentation to Generate Quantifier Ion-MS/MS Spectrum of a Peptide Having a Carbamate-Based Label at the C Terminus

Peptide 1 ATNSQFLR (SEQ ID No. 1) having one carbamate containing (poly)label (2) bound via an amide bond at the C terminus, prepared according to Reference Example 3, was investigated via LCMS/MS. The MS/MS spectrum is shown in FIG. 4.

It was shown that, unlike a peptide without (poly)label (see Comparative Example 1, FIG. 1), first, the unstable chemical analyte (HO—CO—NH—CH₂—CH₂—N⁺(CH₃)₃ with 147 Da) broke apart from the peptide-(poly)label construct and then it underwent a water-loss to generate a stable quantifier ion with MH⁺¹=129 Da (O═CNH—CH₂—CH—N⁺(CH₃)₃).

### Example 4: Multi-Fragmentation Event on Individual Analytes-Adenine-Based (poly)label at C Terminus and N-Terminus

A peptide-(poly)label construct having multiple copies of a “moiety” was expected to undergo multiple fragmentation events on each individual peptide. To demonstrate this, a peptide 1 ATNSQFLR (SEQ ID No. 1) coupled with a (poly)label at the C-terminus and a (poly)label at the N-terminus terminus, was synthesized according to Reference Example 4 and analyzed by LCMS/MS. The MS/MS spectrum is shown in FIG. 5. Since this peptide-(poly)label construct had adenine-based (poly)label at each terminus, the detection of a peptide-(poly)label fragment lacking both “quantifier moieties” indicated that a multi-fragmentation event took place on the individual peptide-(poly)label construct.

### Example 5: Multi-Fragmentation Event on Individual Analytes-APG-Based (poly)label at C Terminus and at N Terminus

A peptide-(poly)label construct having multiple copies of a certain “moiety” was expected to undergo multiple fragmentation events on each individual peptide. To demonstrate this, peptide 1 ATNSQFLR (SEQ ID No. 1) was derivatized with 1 APG-containing (poly)label at the N terminus and one APG containing (poly)label at the N terminus. i.e. two glutamic acids, each carrying a APG group coupled to the free COOH group of the glutamic acid by an amide bond, were coupled, and analyzed by LCMS/MS. The MS/MS spectrum is shown in FIG. 6. Since this peptide-(poly)label construct had two APG-based (poly)labels, the detection of a peptide-(poly)label fragment lacking both “quantifier moieties” indicated that a multi-fragmentation event took place on the individual peptide-(poly)label construct.

### Example 6: Multi-Fragmentation Event on Individual Analytes-Carbamate Based (poly)label at C Terminus and N Terminus

A peptide-(poly)label construct having multiple copies of the “moiety” was expected to undergo multiple fragmentation events on each individual peptide. To demonstrate this, a peptide 1 ATNSQFLR (SEQ ID No. 1) harboring a (poly)label with a carbamate residues [(poly)label tag (1)] at the C terminus and N terminus was synthesized according to Reference Example 3 and analyzed by LC-MS/MS. The MS/MS spectrum is shown in FIG. 7. Since this peptide-(poly)label construct had two carbamate residues, the detection of a peptide-(poly)label fragment lacking both “quantifier moieties” indicated that a multi-fragmentation event took place on the individual peptide-(poly)label construct.

### Example 7: Signal Intensity or Fragmentation Efficiency Increase with Multinumber of Adenine-Based (poly)label

Peptide 1 ATNSQFLR (SEQ ID No. 1) having one adenine residue bound to the C terminus was synthesized according to Reference Example 4 based on (poly)label tag (3) and a polypeptide ATNSQFLR (SEQ ID No. 1) having two adenine residues bound to the C terminus was also synthesized according to Reference Example 4 based on (poly)label tag (4) and investigated, based on LC-MS/MS data in that the relative Selected Reaction Monitoring (SRM) intensities were compared; the results are graphically shown in FIG. 8.

$$Relative SRM intensity = SRM {Intensity}_{( Unmodified Peptide )} / SRM {Intensity}_{( Pep . \sim n ( ( poly ) label ) )}$$

It was shown that due to the presence of multiple units of the “moiety”, and highly selective fragmentation of (poly)label containing peptides, the SRM intensity of the quantifier ion increased. It was further shown that with increasing number of (poly)labels per analyte, relative fragmentation efficiency increased, indicating multiple fragmentation events per individual peptide molecule as shown in FIG. 10.

$$Fragmentation efficiency ( \% ) = ( Product ion intensity / Precursor ion intensity ) * 100$$

$$Relative fragmentation efficiency = Fragmentation \text{} {Efficiency}_{( Pep . \sim n ( ( poly ) label ) )} / Fragmentation {Efficiency}_{( Unmodified Peptide )}$$

### Example 8: Signal Intensity or Fragmentation Efficiency Increase with Multinumber of APG-Based (Poly)Label

Peptide 1 ATNSQFLR (SEQ ID No. 1) having one APG residue bound to the C terminus and a peptide 1 ATNSQFLR (SEQ ID No. 1) having two APG residues bound to the C terminus investigated based on LC-MS/MS data in that the relative Selected Reaction Monitoring (SRM) intensities were compared; the result is graphically shown in FIG. 9.

$$Relative SRM intensity = SRM {Intensity}_{( Unmodified Peptide )} / SRM {Intensity}_{( Pep . \sim n ( ( poly ) label ) )}$$

It was shown that due to the presence of multiple units of the “moiety”, and highly selective fragmentation of (poly)label containing peptides, the SRM intensity of the quantifier ion increased. It was further shown that with increasing number of (poly)labels per analyte, relative fragmentation efficiency increased, indicating multiple fragmentation events per individual peptide molecule as shown in FIGS. 10 and 11.

$$Fragmentation efficiency ( \% ) = ( Product ion intensity / Precursor ion intensity ) * 100$$

$$Relative fragmentation efficiency = Fragmentation \text{} {Efficiency}_{( Pep . \sim n ( ( poly ) label ) )} / Fragmentation {Efficiency}_{( Unmodified Peptide )}$$

### Example 9: Investigation of Higher Numbers of Quantifier Moieties Per Analyte

### 9a: Relative SRM Signal Intensity for Carbamate Based (Poly)Label

To investigate whether a high number (>2) of (poly)labels can be accommodated per analyte, 1×-, 2×-, 4×-, and 6×-(poly)label-Peptide constructs based on carbamate were synthesized and investigated by LCMS and consequently in that the relative Selected Reaction Monitoring (SRM) intensities were compared; the result is graphically shown in FIG. 12.

$$Relative SRM intensity = SRM {Intensity}_{( Unmodified Peptide )} / SRM {Intensity}_{( Pep . \sim n ( ( poly ) label ) )}$$

In this example, roughly 2.5 fold higher SRM intensity was measured with peptide-6×((poly)label) construct.

### 9b: Relative Fragmentation Efficiency for Carbamate Based (Poly)Label

For the 1×-, 2×-, 4×-, and 6×-(poly)label-Peptide constructs based on carbamate of 9a, the Relative fragmentation efficiency was calculated:

$$Fragmentation efficiency ( \% ) = ( Product ion intensity / Precursor ion intensity ) * 100$$

$$Relative fragmentation efficiency = Fragmentation \text{} {Efficiency}_{( Pep . \sim n ( ( poly ) label ) )} / Fragmentation {Efficiency}_{( Unmodified Peptide )}$$

The results are graphically shown in FIG. 13. Unlike Adenine and APG-based (poly)labels, carbamate-based (poly)label provided only modest fragmentation efficiency increase.

### Example 10: Derivatization of Tryptic Peptides with NHS Ester Containing 2×- and 4×-(Poly)Labels

To demonstrate applicability of the (poly)label concept to tryptic peptides, three different synthetic tryptic peptides (Peptide 1 of Sequence ID No. 1, Peptide 2 of SEQ ID No. 2 and Peptide 3 of SEQ ID No. 3) were reacted at their N termini with NHS ester reactive group containing 2×- and 4×-(poly)label based on carbamate [(poly)label tag (1), (poly)label tag (2), see Reference Example 3], and after derivatization peptides were analyzed by LCMS. Relative fragmentation efficiency was calculated:

$$Relative fragmentation efficiency = Fragmentation \text{} {Efficiency}_{( Pep . - n \sim 4 x ( ( poly ) label ) )} / Fragmentation {Efficiency}_{( Pep . - n \sim 2 x ( ( poly ) label ) )}$$

The results are graphically shown in FIG. 14. For all three peptides, the 4×-(poly)label peptide construct yielded almost 4-fold fragmentation efficiency. It is very plausible to expect that in case of 100% derivatization of peptides, 4-fold higher fragmentation efficacy would be also reflected on SRM intensities.

### SHORT DESCRIPTION OF THE FIGURES

FIG. 1 shows the MS/MS spectrum of a model peptide (ATNSQLFR). Letters above the peaks designate the peptide fragments according to peptide fragmentation nomenclature.

FIG. 2 shows the MS/MS spectrum of synthetic peptide-(poly)label construct (ATNSQLFR-P). “P” designates (Adenine-based) (poly)label construct. “Quantifier ion” means here adenine (C5H6N5) with MH⁺¹ (Da)=136.

FIG. 3 shows the MS/MS spectrum of synthetic peptide-(poly)label construct (ATNSQLFR-P). “P” designates an APG-based (poly)label construct. ATNSQLFR~P* designates the peptide-(poly)label construct lacking the moiety, wherein the “moiety” was PG (Proline-Glycine, C7H12N2O3, MH⁺¹ (Da)=172).

FIG. 4 shows the MS/MS spectrum of synthetic peptide-(poly)label construct (ATNSQLFR-P). “P” designates the Carbamate-based (poly)label construct. ATNSQLFR~P* designates the peptide-(poly)label construct lacking the quantifier ion, wherein the “moiety” was O═CNH—CH₂—CH—N⁺(CH₃)₃ with MH⁺¹ (Da)=129.

FIG. 5 shows the MS/MS spectrum of peptide-(poly)label construct containing (poly)labels at C and N terminus of the construct. “P~ATNSQLFR~P” designated intact Peptide (poly)label having two quantifier ions. P*~ATNSQLFR~P designated Peptide (poly)label from which the one quantifier ion fell off. P*~ATNSQLFR~P* designated peptide-(poly)label construct from which two quantifier ions fell off. Detection of the peptide-(poly)label construct (P*~ATNSQLFR~P*) demonstrated that the multi-fragmentation event took place on the individual peptide analyte. “Quantifier ion” means here adenine (C5H6N5) with MH⁺¹ (Da)=136.

FIG. 6 shows the MS/MS spectrum of peptide-(poly)label construct containing APG-based (poly)labels at C and N terminus of the construct. P*~ATNSQLFR~P designated Peptide (poly)label construct from which the one quantifier ion fell off. P*~ATNSQLFR~P* designated the peptide-(poly)label construct from which two quantifier ions fell off. Detection of peptide-(poly)label construct (P*~ATNSQLFR~P*) demonstrated that multi-fragmentation event takes place on individual peptide analytes, wherein the “moiety” was PG (Proline-Glycine, C7H12N2O3, MH⁺¹ (Da)=172).

FIG. 7 shows the MS/MS spectrum of carbamate based Peptide-2× ((poly)label) construct. Fragmentation was optimized to detect intact ATNSQLFR ~P~P, fragment ATNS-QLFR~P~P* and ATNSQLFR~P*~P* ions. ATNSQLFR~P~P designates the intact Peptide-2× ((poly)label) construct, ATNSQLFR~P~P* designates the remnant fragment ion from which a 147 Da fragment fell off and ATNSQLFR~P*~P* is the remnant fragment ion lacking 2×147 Da, wherein the “moiety” was O═CNH—CH₂—CH—N⁺(CH₃)₃ with MH⁺¹ (Da)=129.

FIG. 8 shows relative SRM signal intensity of synthetic peptide in unmodified state and as 1×- and 2×-(poly)label constructs for adenine-based (poly)label. SRM intensities were normalized to SRM intensity of unmodified peptide. SRM intensity of the unmodified peptide was monitored with highest abundant fragment ion (y6 ion, 764.6 m/z), and (poly)labeled peptides were monitored with “quantifier ion” having MH⁺¹ (Da)=136 (adenine, C5H6N5).

FIG. 9 shows relative SRM signal intensity of synthetic peptide in unmodified state and as 1×- and 2×-(poly)label constructs for APG-based (poly)label. SRM intensities were normalized to SRM intensity of unmodified peptide. SRM intensity of the unmodified peptide was monitored with highest abundant fragment ion (y6 ion, 764.6 m/z), and (poly)labeled peptides were monitored with “quantifier ion” having MH⁺¹ (Da)=172 (PG, roline-Glycine, C7H12N2O3).

FIG. 10 shows relative fragmentation efficiency of synthetic peptide in unmodified state and as 1×- and 2×-(poly)label constructs for adenine-based (poly)label. Individual fragmentation efficiency was normalized to fragmentation efficiency of unmodified peptide.

FIG. 11 shows relative fragmentation efficiency of synthetic peptide in unmodified state and as 1×- and 2×-(poly)label constructs for APG-based (poly)label. Individual fragmentation efficiency was normalized to fragmentation efficiency of unmodified peptide.

FIG. 12 shows relative SRM signal intensity of synthetic unmodified, carbamate-based 1×-, 2×-, 4×-, and 6×-(poly)label containing peptide constructs: SRM intensities were normalized with SRM intensity of unmodified peptide. SRM intensities of the unmodified peptide was monitored with highest abundant fragment ion (y6 ion, 764.6 m/z), and (poly)labeled peptides were monitored with “quantifier ion” having MH⁺¹ (Da)=129 (O═CNH—CH₂—CH—N⁺(CH₃)₃).

FIG. 13 shows relative fragmentation efficiency of carbamate-based 1×-, 2×-, 4×-, and 6×-(poly) label containing peptide constructs. Individual fragmentation efficiencies were normalized to fragmentation efficiency of unmodified peptide.

FIG. 14 shows relative fragmentation efficiency of derivatized tryptic peptides with NHS-ester containing carbamate-based 2×- and 4×-((poly)labels). For each individual peptide (poly)label construct fragmentation efficiency was normalized to fragmentation efficiency of Peptide-2×((poly)label).

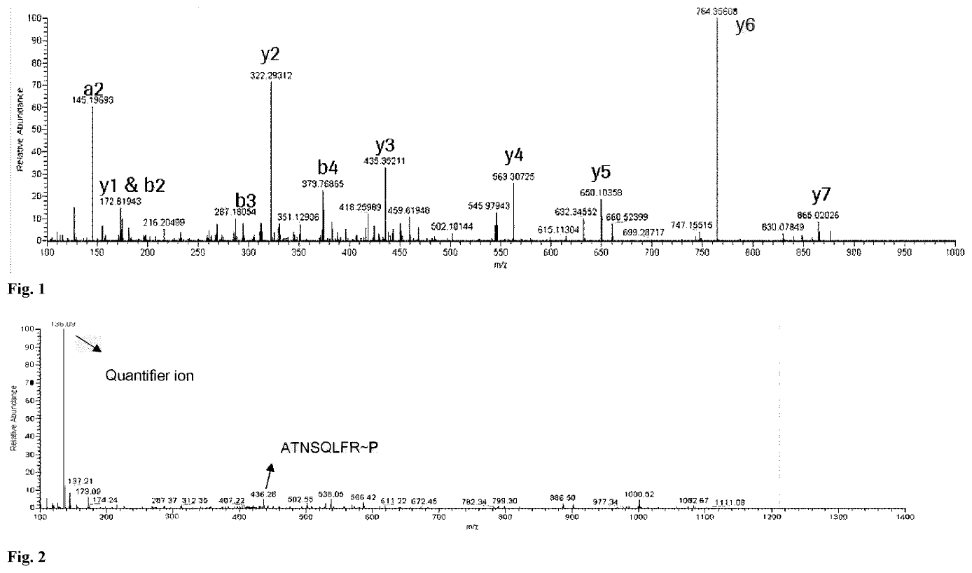

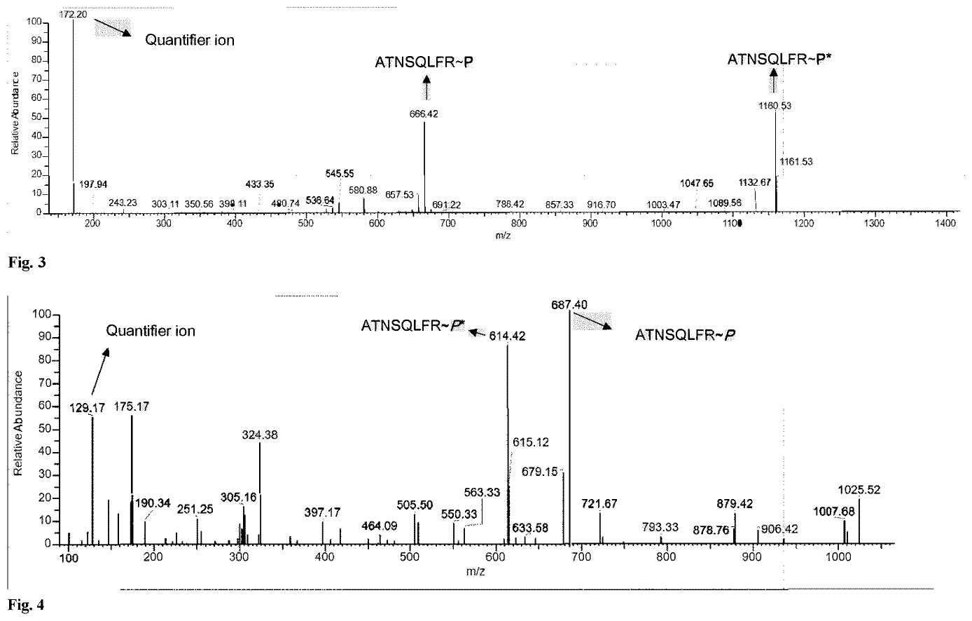

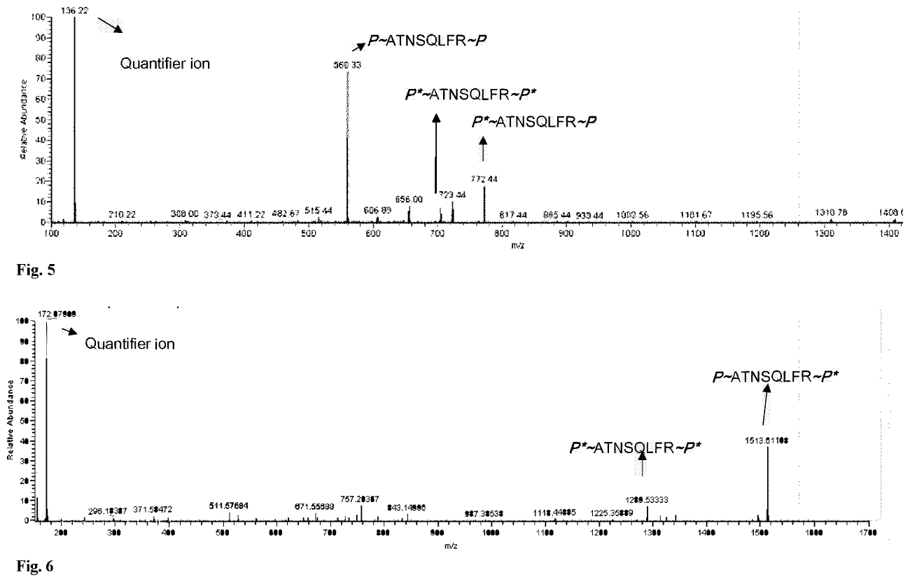

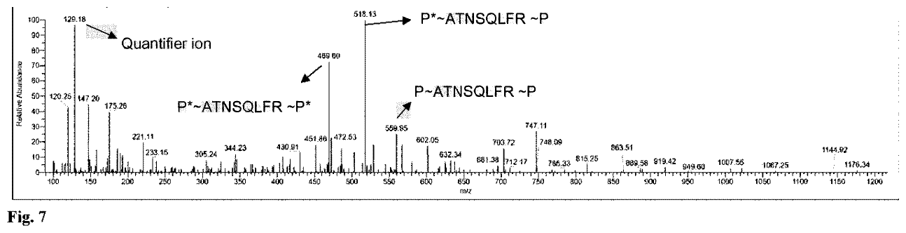

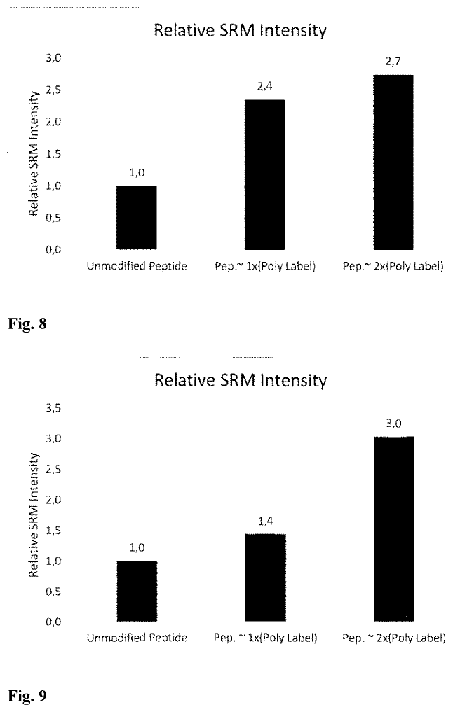

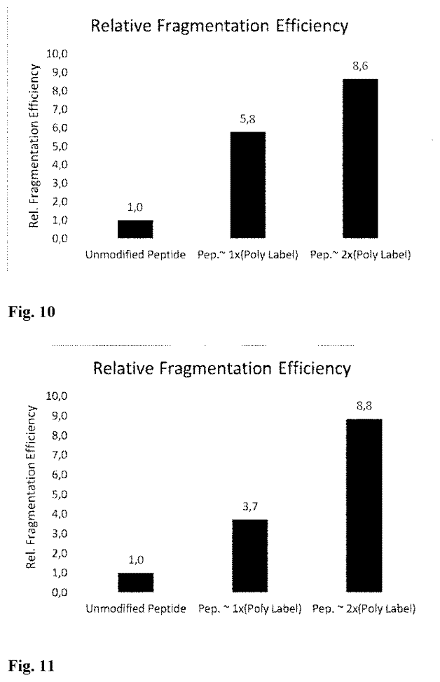

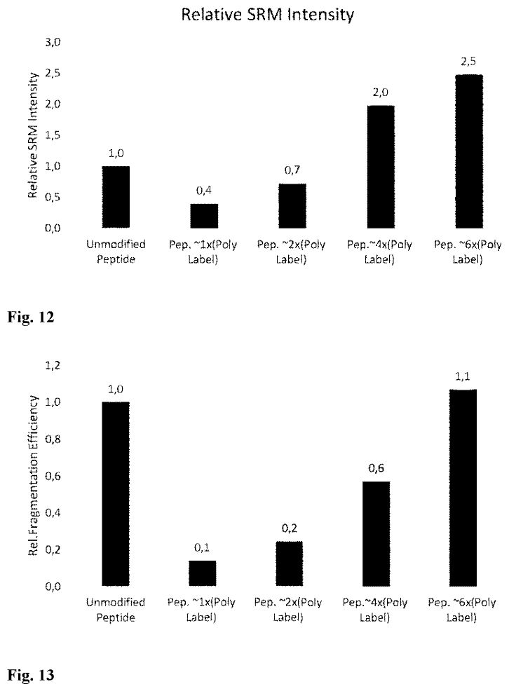

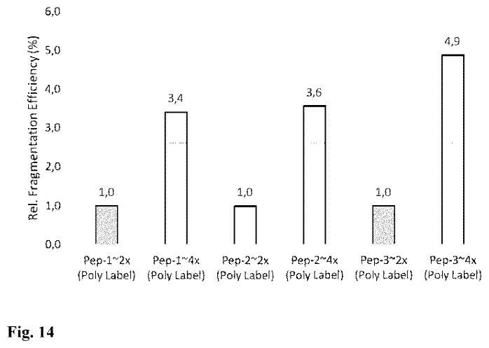

### CITED LITERATURE

Hahne et al. 2013: Hahne, H., Pachl, F., Ruprecht, B. et al. DMSO enhances electrospray response, boosting sensitivity of proteomic experiments. Nat Methods 10, 989-991 (2013)
Mirzaei et al. 2006: Mirzaei, H.; Regnier, F. Enhancing electrospray ionization efficiency of peptides by derivatization. Anal. Chem. 2006, 78, 4175-4183

1. (canceled)

2. The method of claim 10, wherein the (poly)label has a structure of formula (Ia) 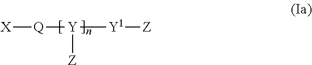
wherein
n is an integer selected from the range of from 1 to 20;
Q is absent or is a linker unit;
X is a reactive group;
Y is a 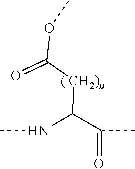
group, or a 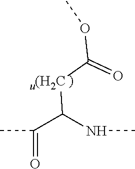
group, wherein the dotted line at the oxygen atom indicates the bond to Z and u is either one or two, or
a 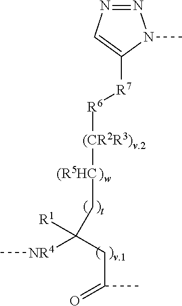
group, or a 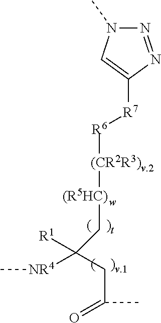
group,
or a 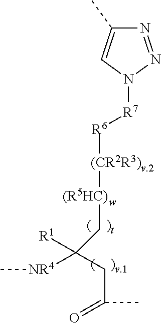
group, or a 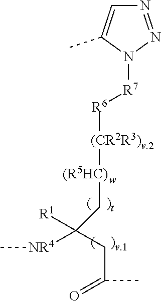
group, wherein the dotted line at the N atom or at the C atom in the triazole ring represent the bond to Z,
t is zero or 1;
v.1 is zero or an integer from the range of 1 to 4;
v.2 is an integer from the range of 1 to 10;
W is zero or 1;
R¹ is a hydrogen atom or a C1-C5 straight or branched alkyl group;
R², R³ are independently from each other and independently for each of the v.2 units selected from hydrogen atom and C1-C5 straight or branched alkyl group;
R⁴ is a hydrogen atom or a C1-C5 straight or branched alkyl group;
R⁵ is a hydrogen atom;
R⁶ is absent or a —C(═O)NH— group or a C(═)O-group or a substituted or unsubstituted C6 to C10 arylene, wherein the one or more substituents are selected from hydrogen atom, halogen atom and functional group;
R⁷ is absent or selected from the group consisting of branched or unbranched C1-C5 alkylene, —O—C1 to C5 alkylene, wherein the C1 to C5 alkylene is branched or unbranched, and —S—C1 to C5 alkylene, wherein the C1 to C5 alkylene is branched or unbranched;
or R⁴ and R⁵ together form a five or six membered heteroalkyl ring, which includes the nitrogen atom of NR⁴ as part of the ring structure;
wherein the dotted line at the NH or NR⁴ for each Y group represents the bond to Q, to the next [Y—Z] unit or to Y¹, the dotted line at the C(═O) for each group represents the bond to the next [Y—Z] unit, to Y¹ or to Q;
Y¹ is a 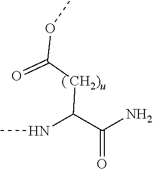
group or a 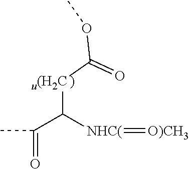
group, wherein the dotted line at the oxygen atom indicates the bond to Z and u is either one or two, or
a 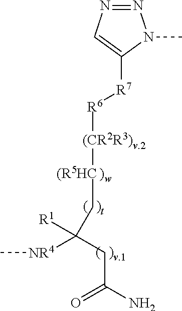
group, or a 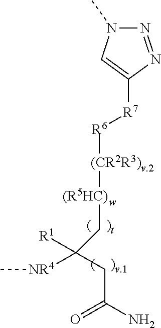
group,
or a 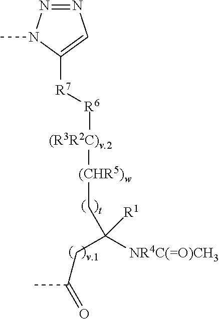
group, or a 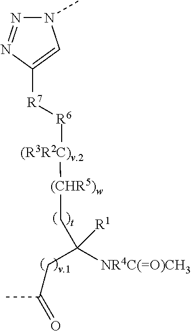
group,
or a 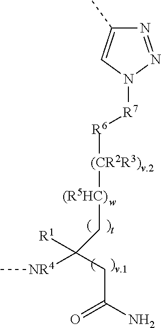
group, or a 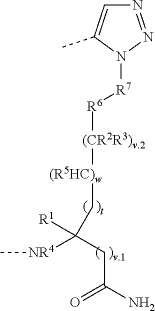
group,
or a 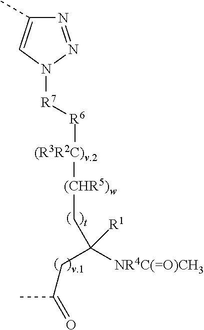
group, or a 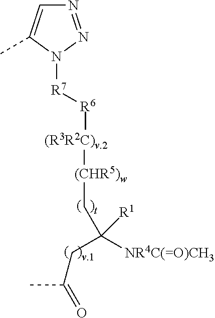
group,
wherein the dotted line at the N atom or at the C atom in the triazole ring represent the bond to Z, and t, v.1, v.2, w and R¹ to R⁵ have the same meaning as above for Y;
wherein the dotted line at the NH or NR⁴ or at the C(═O) for each Y¹ group represents the bond to the next [Y—Z] unit;
Z is a moiety selected from the group consisting of
nucleoside with a nucleobase selected from the group consisting of adenine, cytosine, thymine, guanine and uracil; and
carbamate group —O—C(═O)—NH—(CH₂)_{z}—N⁺(Z³)₃, or —NH—C(═O)—O—(CH₂)_{z}—N⁺(Z³)₃, wherein z is an integer selected from the range of from 1 to 20, and Z³ is a C1 to C5 alkyl group; and
tripeptide Z¹-Proline-Z², wherein Z¹ and Z² are independently from each other an amino acid selected from the group consisting of alanine, glycine, valine, leucine, iso-leucine and phenyl alanine.

3. The method of claim 10, wherein the (poly)label has a structure of formula (Ia), wherein n is an integer selected from the range of from 1 to 20; X is a hydrogen atom; Q is absent,
Y is a 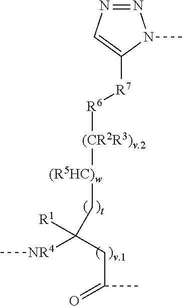
group, or a 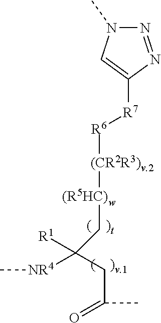
group,
or a 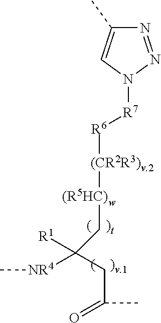
group, or a 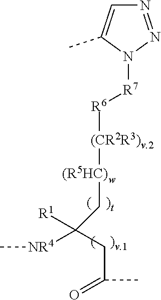
group;
wherein the dotted line at the NR⁴ represents the bond to Q or to the next [Y—Z] unit respectively, the dotted line at the C(═O) represents the bond to the next [Y—Z] unit or to Y¹ respectively and the dotted line at the N atom or the C atom in the triazole ring represent the bond to Z and t, v.1, v.2, w and R¹ to R⁵ have the same meaning as indicated above in claim 2;
Y¹ is a 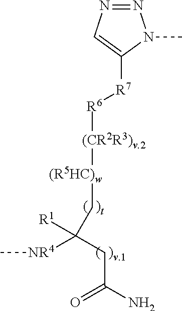
group, or a 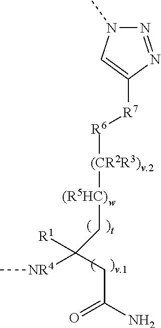
group,
or a 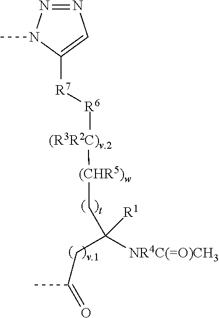
group, or a 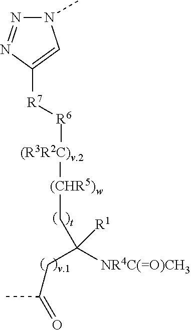
group,
or a 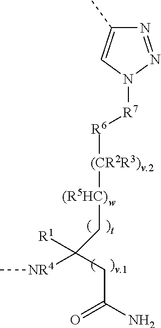
group, or a 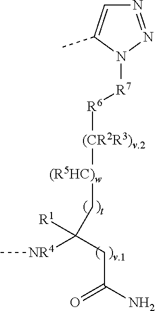
group,
or a 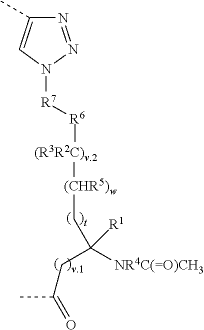
group, or a 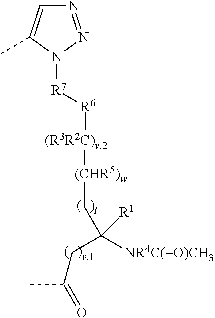
group;
wherein the dotted line at the NR⁴ or at the C(═O) represents the bond to the next [Y—Z] unit, the dotted line at the N atom or at the C atom in the triazole ring represent the bond to Z, and t, v.1, v.2, w and R¹ to R⁵ have the same meaning as indicated above in claim 2;
and
Z is a nucleoside with a nucleobase selected from the group consisting of adenine, cytosine, thymine, guanine and uracil.

4. The method of claim 10, wherein the (poly)label has a structure of formula (Ia), wherein n is an integer selected from the range of from 1 to 20; Q is a linker unit, X is a reactive group;
Y is a 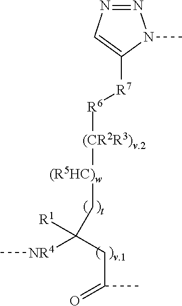
group, or a 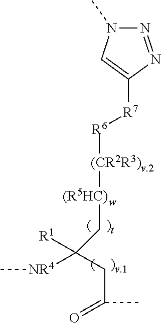
group,
or a 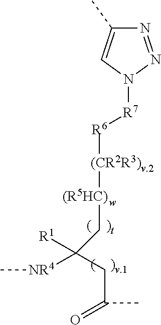
group, or a 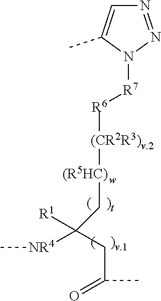
group,
wherein the dotted line at the NR⁴ represents the bond to Q or to the next [Y—Z] unit respectively, the dotted line at the C(═O) represents the bond to the next [Y—Z] unit or to Y¹ respectively and the dotted line at the N atom or at the C atom in the triazole ring represent the bond to Z and t, v.1, v.2, w and R¹ to R⁵ have the same meaning as indicated above in claim 2;
Y¹ is a 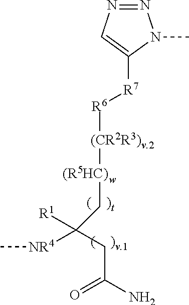
group, or a 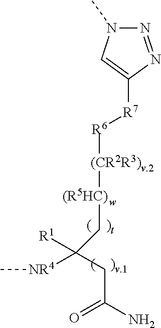
group,
or a 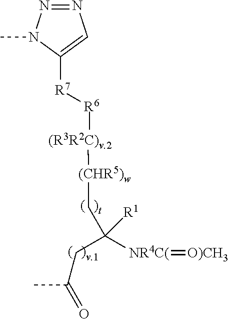
group, or a 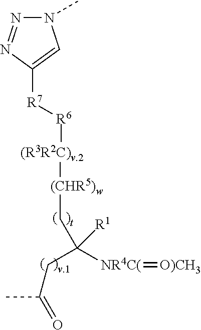
group,
or a 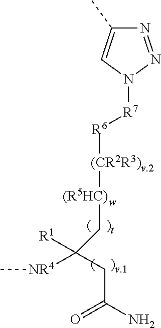
group, or a 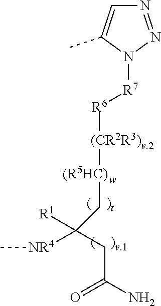
group,
or a 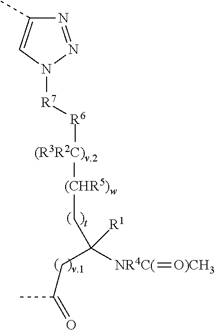
group, or a 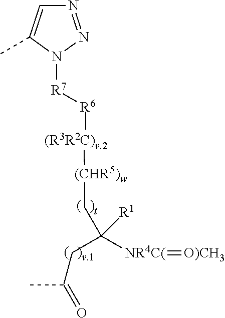
group;
wherein the dotted line at the NR⁴ or at the C(═O) represents the bond to the next [Y—Z] unit, the dotted line at the N atom or at the C atom in the triazole ring represent the bond to Z, and t, v.1, v.2, w and R¹ to R⁵ have the same meaning as indicated above in claim 2;
and
Z is a carbamate group —O—C(═O)—NH—(CH₂)_{z}—N⁺(Z³)₃ or —NH—C(═O)—O—(CH₂)_{z}—N⁺(Z³)₃, wherein z is an integer selected from the range of from 1 to 10, and Z³ is a C1 to C5 alkyl group.

5. The method of claim 10, wherein the (poly)label has a structure of formula (Ia), wherein n is an integer selected from the range of from 1 to 20; Q is absent or is a linker
unit; X is a hydrogen atom or a reactive group;
Y is a 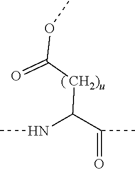
group, wherein the dotted line at the oxygen atom indicates the bond to Z and u is one or two, the dotted line at the NH represents the bond to Q or to the next [Y—Z] unit respectively, the dotted line at the C(═O) represents the bond to the next [Y—Z] unit or to Y¹ respectively;
Y¹ is a 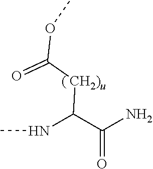
group, or a 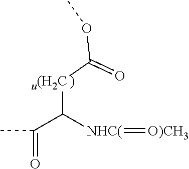
group,
wherein the dotted line at the oxygen atom indicates the bond to Z and u is one or two, and the dotted line at the NH or at the C(═O) represents the bond to the next [Y—Z] unit;
and
Z is a tripeptide Z¹-Proline-Z², wherein Z¹ and Z² are independently from each other an amino acid selected from the group consisting of alanine, glycine, valine, leucine, isoleucine, and phenyl alanine.

6. The method of claim 10, wherein in the (poly)label structure of formula (I) n and m are both zero, Q is absent and X is a hydrogen atom, the (poly)label having a structure of formula (Ib)
Z¹-Proline-Z² (Ib)
wherein Z¹ and Z² are independently from each other an amino acid selected from the group consisting of alanine, glycine, valine, leucine, iso-leucine, and phenyl alanine.

7. The method of claim 10, wherein Q is a linker, which comprises a (C1-C5 alkylene-O—)ᵣ unit with r being an integer in the range of from 1 to 10 or a C1 to C20 alkanediyl unit or a C1 to C20 alkanediyl-heteroaryl unit or a (C1-C5 alkanediyl)-O—(C1-C5 alkanediyl) unit or a C2 to C9 alkanediyl-C(═O) unit or a C2 to C9 alkanediyl-NH unit, or a [NH—C1 to C5 alkanediyl-C(═O)]ₓ unit or a [C(═O)—C1 to C5 alkanediyl-NH]ₓ unit, and/or wherein x is an integer selected from the range of from 1 to 20.

8. The method of claim 10, wherein X is a reactive group selected from the group consisting of isothiocyanate group, isocyanate group, acyl azide group, sulfonyl chloride group, aldehyde group, glyoxal group, epoxide group, oxirane group, carbonate group, aryl halide group, imidoester group, carbodiimide group, anhydride group, fluorophenyl ester group, carboxyl group, HATU ester group, HBTU ester group and NHS ester group.

9. A process for modifying an analyte of interest for obtaining an increased intensity signal in mass spectrometry, the process comprising the steps:
(a) providing at least one (poly)label having a reactive group of the structure (I) 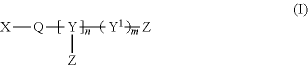
wherein:
m is zero or 1;
n is zero or an integer selected from the range of from 1 to 20;
Q is absent or a linker unit;
X is a reactive group or, if Q is absent, a hydrogen atom;
Y is a linker unit based on an amino acid having a side chain suitable for coupling to Z;
Y¹ is a linker unit based on an amino acid having a side chain suitable for coupling to Z, wherein the amino acid has a blocked carboxylic group or a blocked amino group;
Z is a moiety selected from the group consisting of
nucleoside comprising a nucleobase selected from the group consisting of adenine, cytosine, thymine, guanine and uracil;
tripeptide of structure Z¹-Proline-Z², wherein Z¹ and Z² are independently from each other an amino acid selected from the group consisting of alanine, glycine, valine, leucine, iso-leucine and phenyl alanine; and
carbamate group —O—C(═O)—NH—(CH₂)_{z}—N⁺(Z³)₃ or —NH—C(═O)—O—(CH₂)_{z}—N⁺(Z³)₃, wherein z is an integer selected from the range of from 1 to 10, and Z³ is a C1 to C5 alkyl group;
(b) providing a analyte of interest, which is a polypeptide or a small molecule, and which has a free amino group and/or a free carboxyl group, wherein a free carboxyl group if present is optionally activated;
(c) reacting the at least one (poly)label having a reactive group with the analyte of interest, thereby obtaining a reaction product, wherein the at least one (poly)label is covalently bound to the analyte of interest.

10. A method for determining an analyte of interest by mass spectrometry, the method comprising:
(i) providing a reaction product of the analyte of interest, wherein the reaction product is based on a (poly)label having the structure (I) 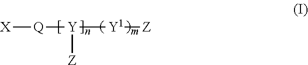
wherein
the (poly)label is covalently bound to the analyte of interest;
m is zero or 1;
n is zero or an integer selected from the range of from 1 to 20;
Q is absent or a linker unit;
X is a reactive group or, if Q is absent, a hydrogen atom;
Y is a linker unit based on an amino acid having a side chain suitable for coupling to Z;
Y¹ is a linker unit based on an amino acid having a side chain suitable for coupling to Z, wherein the amino acid has a blocked carboxylic group or a blocked amino group; and
Z is a moiety selected from the group consisting of
nucleoside comprising a nucleobase selected from the group consisting of adenine, cytosine, thymine, guanine and uracil;
tripeptide of structure Z¹-Proline-Z², wherein Z¹ and Z² are independently from each other an amino acid selected from the group consisting of alanine, glycine, valine, leucine, iso-leucine and phenyl alanine; and
carbamate group —O—C(═O)—NH—(CH₂)_{z}—N⁺(Z³)₃ or —NH—C(═O)—O—(CH₂)_{z}—N⁺(Z³)₃, wherein z is an integer selected from the range of from 1 to 10, and Z³ is a C1 to C5 alkyl group;
(ii) subjecting the reaction product provided in (i) to mass spectrometry; and
(iii) determining the intensity of a fragment corresponding to the MH⁺¹ peak of (unsubstituted) Z or of a water-deprived product thereof in the mass spectrum.

11. A (poly)label having structure of formula (I) 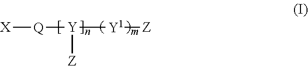
wherein Q, X, Y, Y¹, Y², Z, n and m have the meaning as defined in claim 10.

12. A reaction product comprising a polypeptide and a (poly)label having the general structure (III) 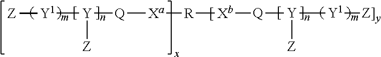
wherein Q, X, Y, Y¹, Y², Z, n and m have the meaning as defined in claim 10 and the indices x, y are either zero or 1 with the condition that at least one of x, y is 1, and wherein R is the remainder of the polypeptide.

13. The method of claim 2, wherein the C terminus of Z² is blocked or amidated.

14. The method of claim 5, wherein the C terminus of Z² is blocked or amidated.

15. The method of claim 6, wherein the C terminus of Z² is blocked or amidated.

16. The method of claim 7, wherein Q is a linker, which comprises a C4 to C7 alkanediyl —C(═O) unit or a C4 to C7 alkanediyl —NH unit.

17. The method of claim 7, wherein Q is a linker, which comprises a hexylene-C(═O) unit or a hexylene-NH unit.

18. The method of claim 7, wherein the C1 to C5 alkanediyl is a C2 to C4 alkanediyl or ethylene.

19. The method of claim 8, wherein X is an NHS ester group.

20. The method of claim 9, wherein the analyte of interest is a polypeptide.
